SEQUENCE LISTING (3 sequences)

SEQ ID NO: 1 - DNA, 21 bp, artificial sequence
  synthetic oligonucleotide
catcatcatcatcatcattga

SEQ ID NO: 2 - DNA, 22 bp, artificial sequence
  synthetic oligonucleotide
ttcgaaaaaaaaatggagttgg

SEQ ID NO: 3 - DNA, 45 bp, artificial sequence
  synthetic oligonucleotide
accggttcaatggtggtgatggtggtgctcgagagatcttttggc

(19) United States
(12) Patent Application Publication
(10) Pub. No.: US 2013/0011909 A1
(43) Pub. Date: Jan. 10, 2013

(54) METHODS AND COMPOSITION TO ENHANCE PRODUCTION OF FULLY FUNCTIONAL P-GLYCOPROTEIN IN PICHIA PASTORIS

(75) Inventor: Ina L. Urbatsch, Lubbock, TX (US)

(73) Assignee: TEXAS TECH UNIVERSITY SYSTEM, Lubbock, TX (US)

(21) Appl. No.: 13/539,367

(22) Filed: Jun. 30, 2012

Related U.S. Application Data
(60) Provisional application No. 61/503,177, filed on Jun. 30, 2011.

(51) Int. Cl. C12N 15/11; G06F 19/10; C12N 1/19

(52) U.S. Cl. 435/254.21; 536/23.1; 435/254.2; 435/254.23; 703/11

(57) ABSTRACT

The present invention provides codon optimization to increase protein production by providing a target gene, wherein the expression of the target gene is to be optimized; determining one or more low-frequency codons in the target gene; providing a codon usage frequency table; replacing each of the one or more low-frequency codons in the target gene with a corresponding high-frequency codons that code for the same amino acid; and harmonizing the a distribution of codon frequencies to those of the set of highly expressed native gene over an open reading frame in the target gene to form an optimized gene, wherein the optimized gene encodes an amino acid sequence identical to the respective wild-type (native) amino acid sequence.

### CROSS-REFERENCE TO RELATED APPLICATIONS

This application claims priority based on U.S. Provisional Application No. 61/503,177, filed Jun. 30, 2011. The contents of each of which is incorporated by reference in its entirety.

### STATEMENT OF FEDERALLY FUNDED RESEARCH

This invention was made with government support under Grant No W81XWH-05-1-0316 awarded by the Department of Defense. The government has certain rights in the invention.

### TECHNICAL FIELD OF THE INVENTION

The present invention relates in general to the field of protein purification, specifically to compositions of matter and methods of making, isolating and purifying proteins.

### INCORPORATION-BY-REFERENCE OF MATERIALS FILED ON COMPACT DISC

None.

### BACKGROUND OF THE INVENTION

The ability of a drug to reach and penetrate its intended target within the body is critical to its success in treating disease. However, drug efflux proteins such as p-glycoprotein (pgp) actively pump hydrophobic drugs away from target tissues and are linked to low oral absorption and multidrug resistance in chemotherapy. Protein pumps are of increasing interest to the pharmaceutical industry, most importantly based on new draft FDA guidelines requiring knowledge of whether a drug candidate is a substrate or inhibitor of pgp. Current pgp assays are cumbersome, expensive and unreliable.

Multiple drug resistance (MDR) mediated by the human MDR-1 gene product was initially recognized during the course of developing regimens for cancer chemotherapy. A multiple drug resistant cancer cell line exhibits resistance to high levels of a large variety of cytotoxic compounds. Frequently these cytotoxic compounds will have no common structural features nor will they interact with a common target within the cell. Resistance to these cytotoxic agents is mediated by an outward directed, ATP-dependent pump encoded by the MDR-1 gene. By this mechanism, toxic levels of a particular cytotoxic compound are not allowed to accumulate within the cell. MDR-like genes have been identified in a number of divergent organisms including numerous bacterial species, the fruit fly Drosophila melanogaster, Plasmodium falciparum, the yeast Saccharomyces cerevisiae, Caenorhabditis elegans, Leighmania donovanii, marine sponges, the plant Arabidopsis thaliana, as well as Homo sapiens.

U.S. Pat. No. 5,837,536, entitled Expression of Human Multidrug Resistance Genes and Improved Selection of Cells Transduced with Such Genes is directed to a DNA sequence for a human MDR1 gene, which encodes p-glycoprotein, wherein at least one base in a splice region of the DNA encoding p-glycoprotein is changed. Such a mutation prevents truncation of the p-glycoprotein upon expression thereof. There is also provided a method of identifying cells which express the human MDR1 gene in a cell population that has been transduced with an expression vehicle including a human MDR1 gene. The method comprises contacting the cell population with a staining material, such as rhodamine 123, and identifying cells which express the human MDR1 gene based on differentiation in color among the cells of the cell population. This method has allowed identification of retroviral producer clones facilitate MDR gene transfer into primary cells. Repopulating hematopoietic stem cells have been genetically engineered with the human MDR1 gene.

U.S. Pat. No. 5,399,483 entitled Expression Of MDR-Related Gene In Yeast Cell is directed to a yeast host which can express P-glycoprotein, i.e., the product of MDR-related gene, in the cell membrane in the same state as observed in multidrug resistant cells produced by connecting the MDR-related gene which carries multidrug resistance to a yeast expression vector and transforming the yeast with said recombinant vector; a cell membrane fraction containing a substantial amount of P-glycoprotein produced by said yeast and a process for the preparation thereof; and a recombinant vector for expressing the MDR-related gene in a yeast host.

### BRIEF SUMMARY OF THE INVENTION

One embodiment of the present invention provides a method of codon optimization to increase protein production by providing an target gene, wherein the expression of the target gene is to be optimized; determining the target gene codons of the target gene; determining a set of low-frequency codons in the target gene; determining one or more highly expressed genes; determining the codons that encode for each of the one or more highly expressed genes; generating a codon usage table from the codons of the one or more highly expressed genes; determining a set of high-frequency codons from the codon usage table; and replacing one or more low-frequency codons with a high-frequency codon that codes for the same amino acid to form an optimized gene, wherein the optimized gene encodes an amino acid sequence identical to the respective wild-type (native) amino acid sequence. The target gene codes may be a P-glycoprotein, e.g., a MDR3 gene or a MDR1 gene. The one or more low-frequency codons may occur at less than about 18, 17, 16, 15, 14, 13, 12, 11, 10, 9, 8, 7, 6, 5, 4, 3, 2, 1, 0.5, or 0.1% frequency and be at incremental variations thereof. Similarly the optimized gene produces at least a 18, 17, 16, 15, 14, 13, 12, 11, 10, 9, 8, 7, 6, 5, 4 or 3 fold increase in the functional protein compared to the expression of a native gene.

Another embodiment of the present invention provides a method of increasing protein production by providing an target gene, wherein the expression of the target gene is to be optimized; determining the target gene codons of the target gene; determining a set of low-frequency codons in the target gene; determining one or more highly expressed genes; determining the codons that encode for each of the one or more highly expressed genes; generating a codon usage table from the codons of the one or more highly expressed genes; determining a set of high-frequency codons from the codon usage table; replacing one or more low-frequency codons with a high-frequency codon that codes for the same amino acid to form an optimized gene, wherein the optimized gene encodes an amino acid sequence identical to the respective wild-type (native) amino acid sequence; and inserting the optimized gene into a cell. The cells may be yeast cells, e.g., a Pichia pastoris cell or a Saccharomyces cerevisiae cell. The target gene may code for a P-glycoprotein, e.g., a MDR3 gene or a MDR1 gene. The one or more low-frequency codons may occur at less than about 18, 17, 16, 15, 14, 13, 12, 11, 10, 9, 8, 7, 6, 5, 4, 3, 2, 1, 0.5, or 0.1% frequency and be at incremental variations thereof. Similarly the optimized gene produces at least a 18, 17, 16, 15, 14, 13, 12, 11, 10, 9, 8, 7, 6, 5, 4 or 3 fold increase in the functional protein compared to the expression of a native gene.

Another embodiment of the present invention provides an expression optimized vector to increase protein production of a functional protein including an optimized nucleic acid vector encoding a target gene wherein the optimized nucleic acid vector comprises at least one high-frequency codons substituted for at least one corresponding low-frequency codon and wherein the optimized nucleic acid vector encodes an amino acid sequence of the target gene is identical to the respective wild-type (native) amino acid sequence. The target gene may code for a P-glycoprotein, e.g., a MDR3 gene or a MDR1 gene. The one or more low-frequency codons may occur at less than about 18, 17, 16, 15, 14, 13, 12, 11, 10, 9, 8, 7, 6, 5, 4, 3, 2, 1, 0.5, or 0.1% frequency and be at incremental variations thereof. Similarly the optimized gene produces at least a 18, 17, 16, 15, 14, 13, 12, 11, 10, 9, 8, 7, 6, 5, 4 or 3 fold increase in the functional protein compared to the expression of a native gene.

Another embodiment of the present invention provides a method of protein optimization by providing a P-glycoprotein gene, wherein the expression of the P-glycoprotein gene is to be optimized; determining the P-glycoprotein gene codons of the P-glycoprotein gene; determining a set of low-frequency codons in the P-glycoprotein gene, wherein the one or more low-frequency codons occur at less than a 18, 17, 16, 15, 14, 13, 12, 11, 10, 9, 8, 7, 6, 5, 4, 3, 2, 1, 0.5, or 0.1% frequency; determining one or more highly expressed genes; determining the codons that encode for each of the one or more highly expressed genes; generating a codon usage table from the codons of the one or more highly expressed genes; determining a set of high-frequency codons from the codon usage table; and replacing one or more low-frequency codons with a high-frequency codon that codes for the same amino acid to form an optimized P-glycoprotein gene, wherein the optimized P-glycoprotein gene encodes an amino acid sequence identical to the respective wild-type (native) amino acid sequence, wherein the optimized gene produces at least a 18, 17, 16, 15, 14, 13, 12, 11, 10, 9, 8, 7, 6, 5, 4 or 3 fold increase in the functional protein compared to the expression of a native gene.

Another embodiment of the present invention provides an expression optimized cell to increase protein production of a functional protein by a yeast cell comprising an optimized nucleic acid vector encoding a P-glycoprotein gene wherein the optimized nucleic acid vector comprises at least one high-frequency codons substituted for at least one corresponding low-frequency codon, wherein the one or more low-frequency codons occur at less than a 18, 17, 16, 15, 14, 13, 12, 11, 10, 9, 8, 7, 6, 5, 4, 3, 2, 1, 0.5, or 0.1% frequency and wherein the optimized nucleic acid vector encodes an amino acid sequence of the P-glycoprotein gene is identical to the respective wild-type (native) amino acid sequence wherein the optimized gene produces at least a 18, 17, 16, 15, 14, 13, 12, 11, 10, 9, 8, 7, 6, 5, 4 or 3 fold increase in the functional protein compared to the expression of a native gene.

In one embodiment the present invention discloses methods, apparatuses and compositions for the purification of proteins. The inventors realized structural and biochemical studies of mammalian membrane proteins remain hampered by inefficient production of pure protein. One embodiment of the present invention provides codon optimization based on highly expressed Pichia pastoris genes to enhance co-translational folding and production of P-glycoprotein (Pgp), an ATP-dependent drug efflux pump involved in multidrug resistance of cancers. Codon-optimized “Opti-Pgp” and wild-type Pgp, identical in primary protein sequence, were rigorously analyzed for differences in function or solution structure. Yeast expression levels and yield of purified protein from P. pastoris (˜150 mg per kg cells) were about three-fold higher for Opti-Pgp than for wild-type protein. Opti-Pgp conveyed full in vivo drug resistance against multiple anticancer and fungicidal drugs. ATP hydrolysis by purified Opti-Pgp was strongly stimulated about 15-fold by verapamil and inhibited by cyclosporine A with binding constants of 4.2±2.2 μM and 1.1±0.26 μM, indistinguishable from wild-type Pgp. Maximum turnover number was 2.1±0.28 mmol/min/mg and was enhanced by 1.2-fold over wild-type Pgp, likely due to higher purity of Opti-Pgp preparations. Analysis of purified wild-type and Opti-Pgp by CD, DSC and limited proteolysis suggested similar secondary and ternary structure. Addition of lipid increased the thermal stability from Tₘ about 40° C. to 49° C., and the total unfolding enthalpy. The increase in folded state may account for the increase in drug-stimulated ATPase activity seen in presence of lipids.

One embodiment of the present invention provides significantly higher yields of protein in the native folded state, higher purity and improved function establish the value of our gene optimization approach, and provide a basis to improve production of other membrane proteins.

P-glycoprotein (mouse MDR3 gene and human MDR1 gene) was codon-optimized for high level expression in the yeast Pichia pastoris and Saccharomyces cerevisiae. The new nucleotide sequences, named mouse Opti-MDR3 and human Opti-MDR1, encode amino acid sequences identical to the respective wild-type (native) proteins. P. pastoris and S. cerevisiae strains transformed with the codon-optimized genes express at least three-fold higher levels of the mouse MDR3 or human MDR1 proteins enabling large-scale production of fully functional P-glycoproteins.

### BRIEF DESCRIPTION OF THE SEVERAL VIEWS OF THE DRAWINGS

For a more complete understanding of the features and advantages of the present invention, reference is now made to the detailed description of the invention along with the accompanying figures and in which:

FIGS. 1A, 1B and 1C are images of a table comparing codon usage.

FIG. 2A is an image of the restriction site map of the restriction enzyme sites of the Opti-Pgp gene. FIG. 2B is a plot showing the GC content analyzed with GeneOptimizer of the Opti-Pgp gene in a 40 bp window centered at the indicated nucleotide position.

FIG. 3A is an image of the cloning strategy for pLIC-H6 vector and expression in P. pastoris. FIG. 3B is an amino acid and nucleotide sequence alignment of human wild-type MDR1 and Opti-MDR1.

FIGS. 4A-4E are images the protein expression levels and in vivo biological activity of WT- and Opti-Pgp in S. cerevisiae.

FIGS. 5A and 5B are images of the purification and size exclusion chromatography of WT- and Opti-Pgp from P. pastoris.

FIGS. 6A and 6B are images of graphs of stimulation and inhibition of ATPase activity.

FIG. 7 is an image of the CD spectra of WT- and Opti-Pgp. CD spectra of the purified proteins were recorded after buffer exchange by size-exclusion.

FIGS. 8A-8F are images of the Differential Scanning calorimetry of WT- and Opti-Pgp.

FIG. 9 is an image of a graph of the lipid dependence of ATPase activity.

FIG. 10 is an image illustrating determining the sensitivity of WT- and Opti-Pgp to trypsin.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Structural, biochemical and pharmaceutical studies of membrane proteins, especially mammalian proteins, remain hampered by inefficient production of pure protein. The codon optimization achieves three-fold higher yields of pure protein with a quality similar or better than wild-type P-glycoprotein produced from Pichia pastoris yeast.

The present invention generated a codon usage table based on highly expressed genes in P. pastoris and found that codon usage in P. pastoris (and in S. cerevisiae yeast) is significantly more stringent in highly expressed genes, as evident from the larger number of low-frequency codons. Furthermore, there are inverted preferences for certain yeast preferred and higher frequency codons suggesting that preferred codons assigned in currently available databases (e.g. Kazusa database) may not represent the best codon choices for high level expression. The present invention provides a new approach that omitted the 19 rare codons {<1 0% frequency) but to completely harmonize the frequency of codons to those of highly expressed P. pastoris genes, and so to maximize translational efficiency by emulating the host's evolutionarily determined codon usage strategy.

P-glycoprotein (Pgp², also known as multidrug resistance protein MDR1 or ABCB1) is a plasma membrane protein that has the ability to pump a wide range of hydrophobic compounds out of cell and has particular relevance to chemotherapy, because it is able to prevent accumulation of many anti-cancer drugs in cells, thus conferring multidrug resistance (MDR) [1]. Therefore, Pgp has been a target for improving cancer treatment and has also been therapeutic targeted for its role in MDR of HIV, epilepsy, and psychiatric illnesses [5, 6, 7, 8]. Pgp is an ABC transporter that requires the energy from ATP binding and hydrolysis in the nucleotide binding domains (NBDs) to drive drug transport across the membrane. Drug binding to the transmembrane domains (TMDs) typically stimulates ATP hydrolysis in the NBDs [9], while inhibitors may compete with drug binding at the polyspecific drug binding sites and so block transport activity and/or ATP hydrolysis. Pgp, like other ABC transporters, is thought to alternate between an inward-facing, drug-binding competent conformation with the transmembrane domains (TMDs) open to the cytoplasm, and an outward-facing, drug-releasing conformation with the TMDs accessible to the extracellular space [10]. The X-ray structure of this mammalian ABC transporter in the inward-facing conformation at 3.8 Å resolution was solved [11]. Co-crystal structures with two inhibitors provided a first glimpse of the interactions between bound inhibitors and the drug binding site residues. However, much work remains to fully understand the interaction of Pgp with drugs and inhibitors and the molecular mechanism of drug export. For these endeavors, large-scale production of the fully functional protein is essential.

Pgp in its fully active form was expressed in the yeast Pichia pastoris and purified [12, 13]. This yeast grows to very high densities in fermentor cultures providing ample source material. However, the modest expression level of this integral membrane protein still presents a bottleneck to large scale protein production. Analysis of genes highly expressed in the yeast Saccharomyces cerevisiae has revealed a strong relationship between tRNA multiplicity and codon selection [14, 15, 16], suggesting that codon usage bias may be one of the factors that lead to inefficient translation and limit protein production. While effective E. coli strains have been developed to overcome the codon bias problem in that expression platform [17], relatively little has been done to address the problem in P. pastoris [18, 19, 20, 21, 22]. Previous gene optimization procedures were commonly based on the Kazusa codon usage database, but an important limitation is that it does not discriminate between poorly and highly expressed genes. Because translation efficiency of more highly expressed genes may be especially sensitive to codon usage, attention to this aspect of gene sequence may be profitable for maximizing protein expression.

One embodiment of the present invention provides a codon usage table specific for highly expressed genes in P. pastoris and found that codon usage bias for this subgroup is significantly more stringent than the average codon usage of genes present in the Kazusa database and in the recently published P. pastoris genome [23, 24]. The sequence of the Pgp-encoding MDR3 gene was codon-adjusted, taking into account relative codon frequencies for each amino acid, as well as optimizing GC content and controlling for mRNA instabilities and Pgp expression was significantly increased. Previous studies found that silent single nucleotide polymorphisms can alter Pgp function and tertiary structure; therefore it was imperative to ascertain that Opti-Pgp retained its functionality, polyspecific drug interactions and folded state. Opti-Pgp was fully active in vivo in yeast drug resistance and mating assays. Furthermore, the quality of the purified protein was improved as judged by size-exclusion chromatography and by ATP hydrolysis rates. Consistent with its activity, the codon-optimized protein exhibited secondary and tertiary structure similar to wild-type (WT) Pgp based on circular dichroic spectroscopy and differential scanning calorimetry analysis of its thermal unfolding properties, respectively.

n-Dodecyl-β-D-maltopyranoside (DDM) was obtained from Inalco Pharmaceutical (Milan, Italy), and E. coli polar lipid extract from Avanti Polar Lipids (Alabaster, Ala.). Doxorubicin and trypsin were from Sigma-Aldrich (St. Louis, Mo.). FK506 and valinomycin were from AG Scientific (San Diego, Calif.).

Optimization of the Pgp gene—The mouse MDR 3 nucleotide sequence (accession number NM_{—}011076), with all three N-glycosylation sites N83, N87 and N90 replaced by glutamine [25] was optimized. Codon substitutions were based on a usage frequency table we calculated for 30 native genes (15,863 codons) known to be highly expressed in P. pastoris. These include AC01 (Pas_chr1-3_{—}0104), ACS1 (Pas_chr2-1_{—}0767), AOX1 (Pas_chr4_{—}0821, PPU96967); CAT2 (Pas_chr3_{—}0069), CCPI (Pas_chr2-2_{—}0127), CDC19 (Pas_chr2-1_{—}0769), CTAI (Pas_chr2 2_{—}0131), ENO1 (Pas_chr3_{—}0082), FBAI (Pas_chr1-1_{—}0072), FDHI (Pas_chr3_{—}0932), FLD (AF066054), GDH3 (Pas_chr1-1_{—}0107), GPMI (Pas_chr3_{—}0826), GUT2 (Pas_chr3_{—}0579) HSP82 (Pas_chr1-4_{—}0130), ICLI (Pas_chr1-4_{—}0338), ILV5 (Pas_chr1-1_{—}0432), KAR (Pas_chr2-1_{—}0140, AY965684), MDHI (Pas_chr2-1_{—}0238), MET6 (Pas_chr2-1_{—}0160, AY601648), PDII (Pas_chr4_{—}0844, AJ302014), PGKI (Pas_chr1-4_{—}0292), PILI (Pas_chr1-4_{—}0569), RPPO (Pas_chr1-3_{—}0068), SSA3 (Pas_chr3_{—}0230), SSB2 (Pas_chr3_{—}0731), SSCI (Pas_chr3_{—}0365), TDH3 (Pas_chr2-1_{—}0437, also called GAP, PPU62648), TEF2 (Pas_FragB_{—}0052, AY219033), YEF3 (Pas_chr4_{—}0038, also called TEF3, AB018536) ([26, 27, 28, 29, 30] and Mattanovich, unpublished results). Codon usage frequency of the collective open reading frames was calculated using the Entelechon software. For gene optimization, the software Leto was used (version 1.0.11, Entelechon, Germany), imposing the codon usage for the 30 highly expressed genes except in cases where codons were retained in order to preserve desirable restriction enzyme sites.

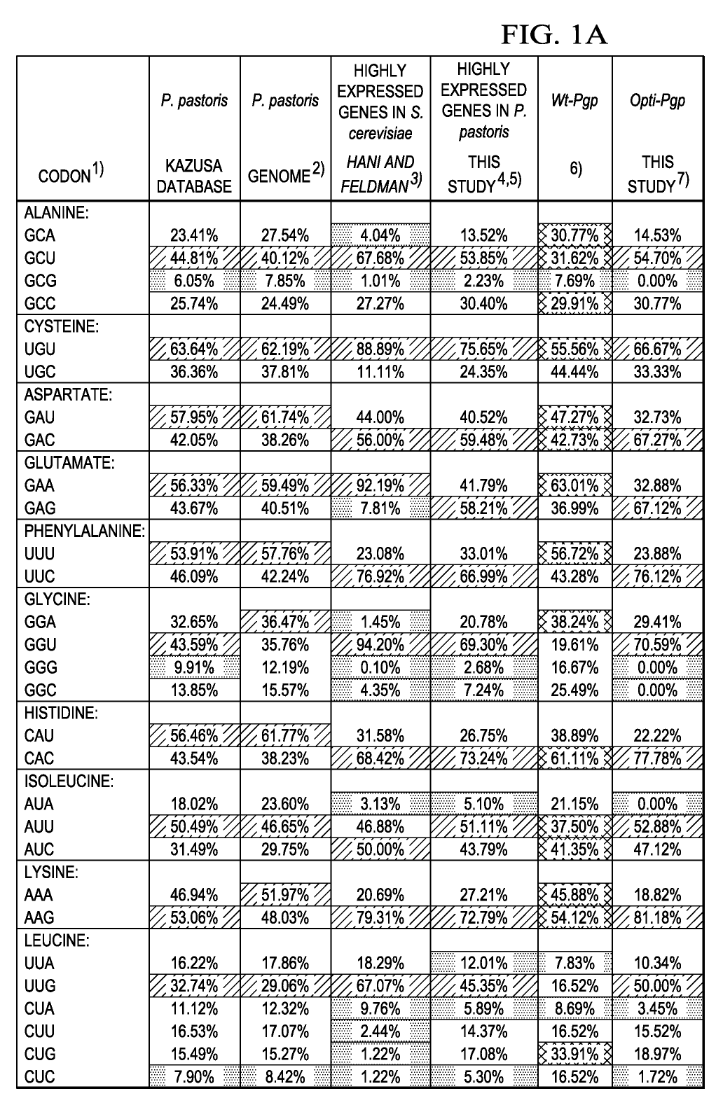

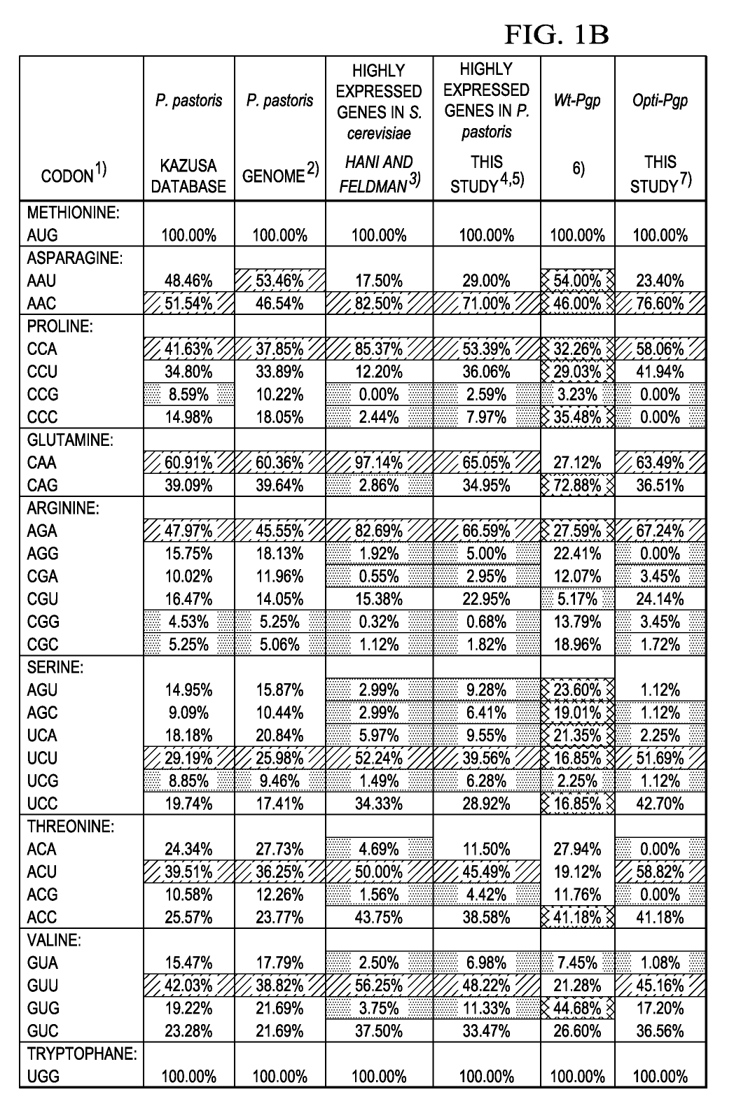

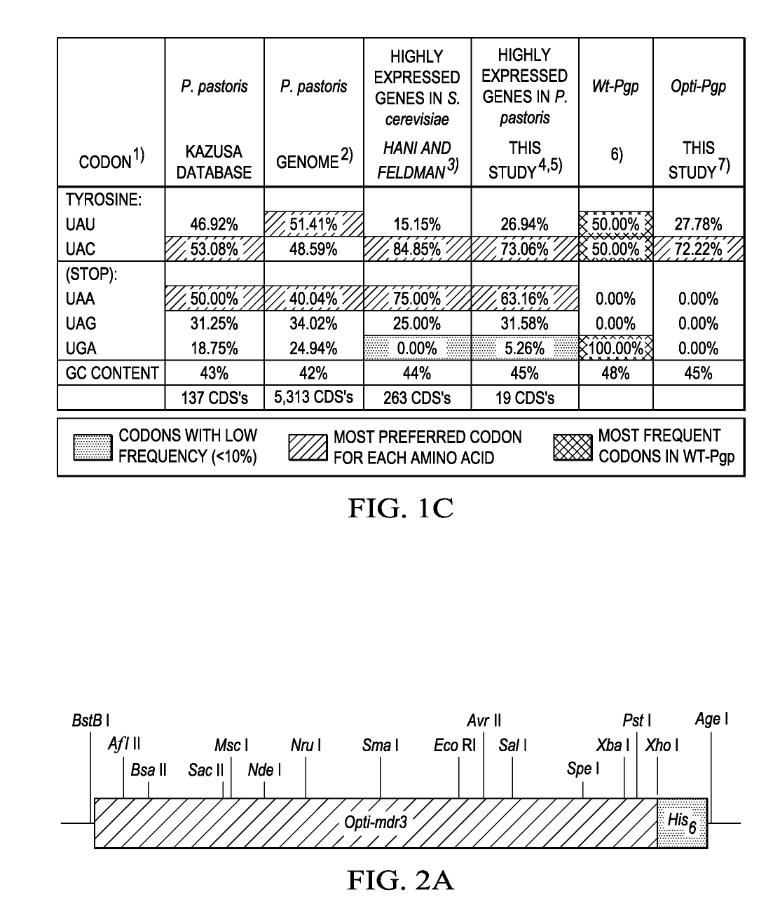

FIGS. 1A, 1B and 1C are images of a table comparing codons. 1) Codons with low frequency (<10%) are highlighted in orange. The most preferred codon for each amino acid is highlighted in light blue. Most frequent codons (and second most frequent, if within 10% of the first) in WT-Pgp are highlighted in light blue. 2) From [23]. Five codons occur at low frequencies in the Kazusa and Genome databases, which do not discriminate between poorly and high expressed genes, e.g. the codons for Ala (GCG), Leu (CUC), Arg (CGG and CGC) and Ser (UGG). Some preferred codons differ between the Kazusa and the Pichia genome databases, namely the codons for Gly, Lys and Asn; this is likely due to the limited number of 13 7 CDS's represented in the former. 3) From [15]. 4) The codon usage analysis was updated to include the 30 most highly expressed genes in P. pastoris based on proteome analysis [26, 27, 28]. Incidentally, all 30 genes are also among the 100 most highly transcribed genes seen in microarrays (Mattanovich, unpublished observations). 5) In highly expressed genes, an additional 18 codons occur at low frequencies, e.g. the codons for Ala (GCA), Gly (GGG and GGC), Ile (AUA), Leu (CUA, CUC and UUA), Pro CCG and CCC), Arg (AGG and CGA), Ser (AGU, AGC and UCA), Thr (ACA and ACG) and Val (GUA and GUG). Comparison of the preferred codon between highly expressed Pichia genes and the Kazusa/genome databases revealed an inverted preference for the Asp codon AAC over AAU, CAC over CAU for His and UUC over UUU for Phe. There was also a strong preference for the Lys codon AAG over AAA, AAC over AAU for Asn, and UAC over UAU for Tyr among highly expressed Pichia genes. Notably, the codon choice for Glu differed between highly expressed genes of the two yeasts with S. cerevisiae showing a clear preference for GAA (92%) whereas P. pastoris has a more balanced distribution of 61:39% between GAA and GAG. 6) The native Pgp revealed extensive codon bias, with pronounced over-representation of codons occurring at low frequency among highly expressed Pichia genes; viz. codons used for Ala (GCG), Gly (GGG, and GGC), Ile (AUA), Leu (CUA and CUC), Pro (CCC and CCG), Arg (AGG, CGA, CGG and CGC), Ser (AGC, AGU, UCA and UCG), Thr (ACG), and Val (GUA). The native gene also under-represented the Pichia higher frequency codons including the preferred codons (compare dark and light blue in columns 4 and 5). For example, the three codons for Ala (GCA, GCU and GCC) are used at about equal frequencies (30-32%) in WT-Pgp whereas highly expressed Pichia genes show a clear preference for GCU (59%) over GCC (31%) and GCA (9%). 7) For gene optimization all low-frequency codons (<8%) were set to zero and the distribution of frequencies adjusted to those of highly expressed Pichia genes. In some cases, desirable restriction enzyme sites required the presence of a low-frequency codon. 8) The C-terminal His 6-tag and STOP codons were provided by the pLIC-H6 vector and were SEQ ID No: 1 CAT CAT CAT CAT CAT CAT TGA.

Furthermore, extended secondary mRNA structure, long range repeats including AT-rich and GC-rich regions and cryptic splice sites were removed and the GC content adjusted to 45%. The Leto software identifies inverted repeats (hairpin stems) with ≦10% mismatches with a distance between inverted repeats (hairpin loops) of at least four nucleotides. For identification of cryptic splice acceptor and donor sites, a hidden Markov model is built in using confirmed splice sites in S. cerevisiae gene sequences retrieved from NCBI Entrez. The software is a multi-objective gene algorithm and takes into account all these parameters at all times to simultaneously optimize over the entire sequence of the gene. Unique restriction sites were introduced to facilitate later genetic manipulations. The optimized “opti-MDR3” gene was synthesized by GeneArt (Regensburg, Germany).

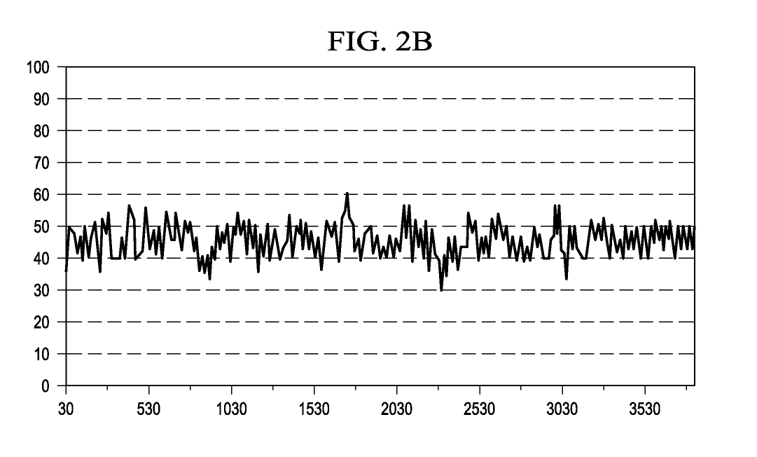

FIG. 2A is an image of the restriction site map of the restriction enzyme sites of the Opti-Pgp gene. The 3,828 bp coding sequence (CDS) of mouse MDR3 is shown with unique restriction enzyme sites; SacII, NruI, AvrII, SalI and SpeI are not present in the Wt sequence, and the gene is flanked by BstBI and XhoI sites. FIG. 2B is a plot showing the GC content analyzed with GeneOptimizer (GeneArt, Germany) of the Opti-Pgp gene in a 40 bp window centered at the indicated nucleotide position.

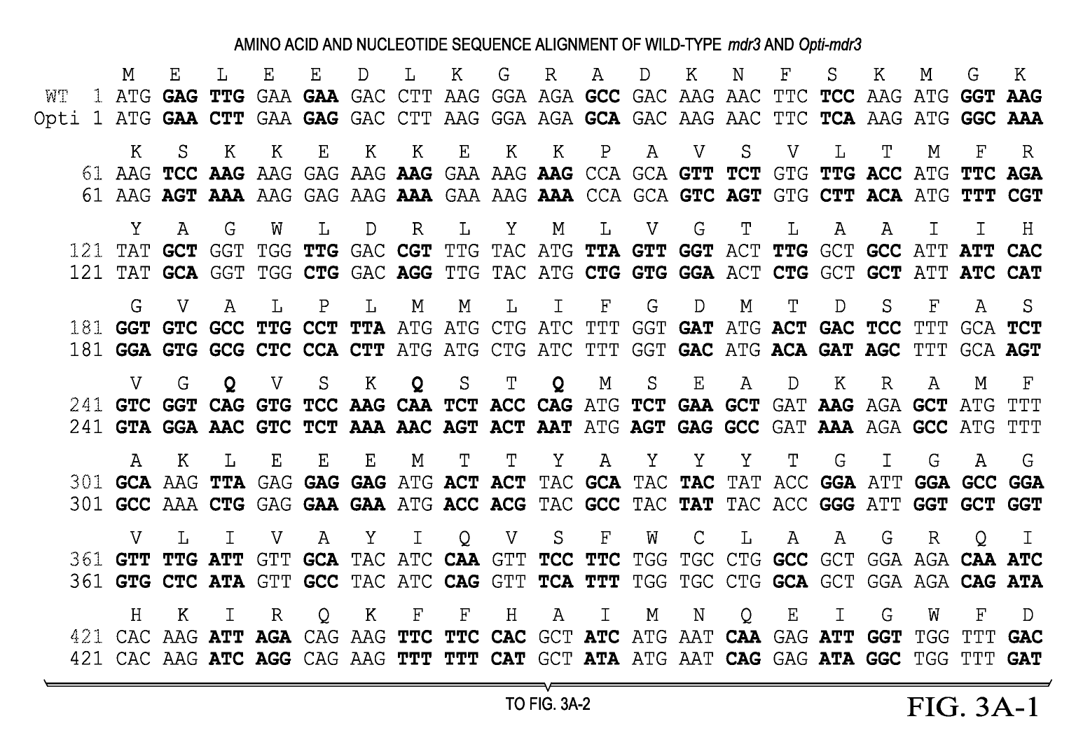

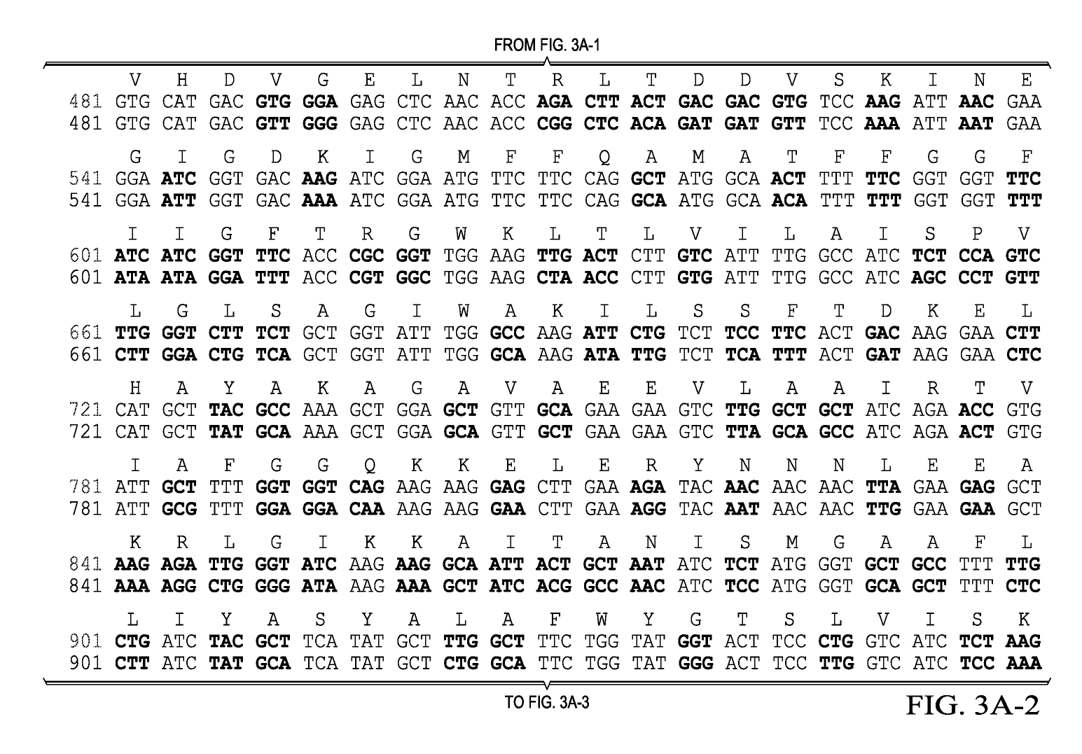

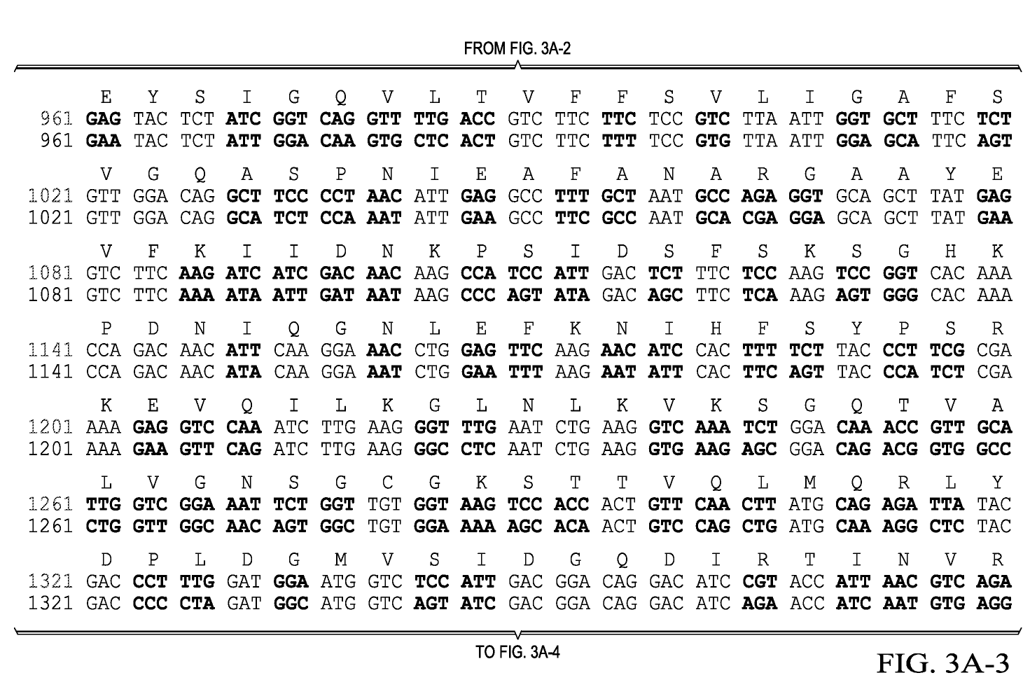

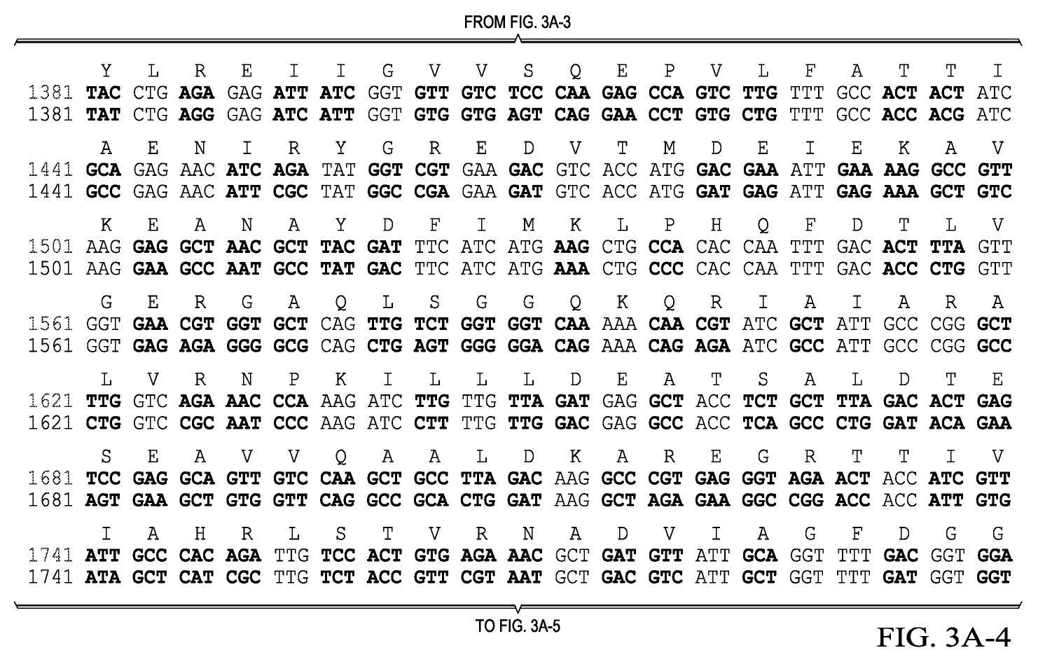

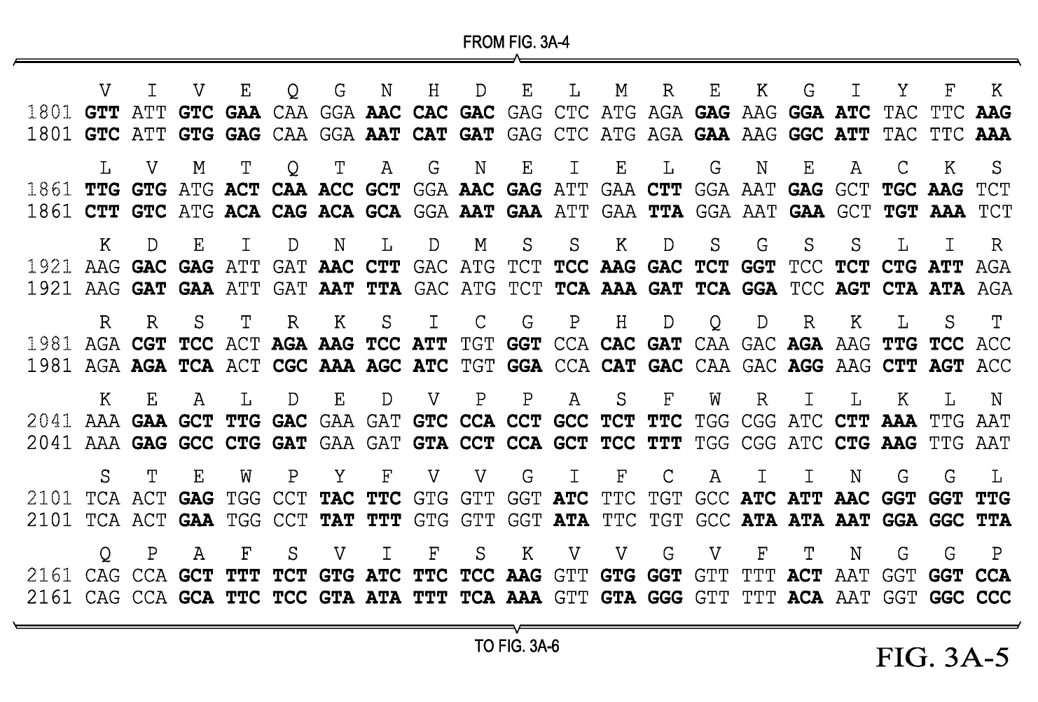

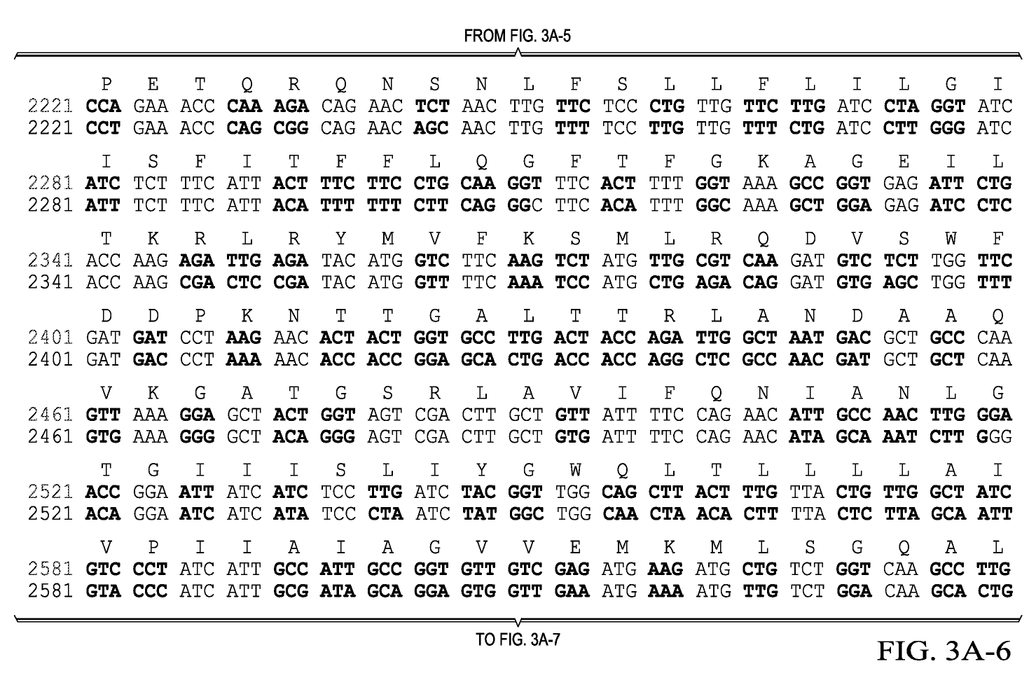

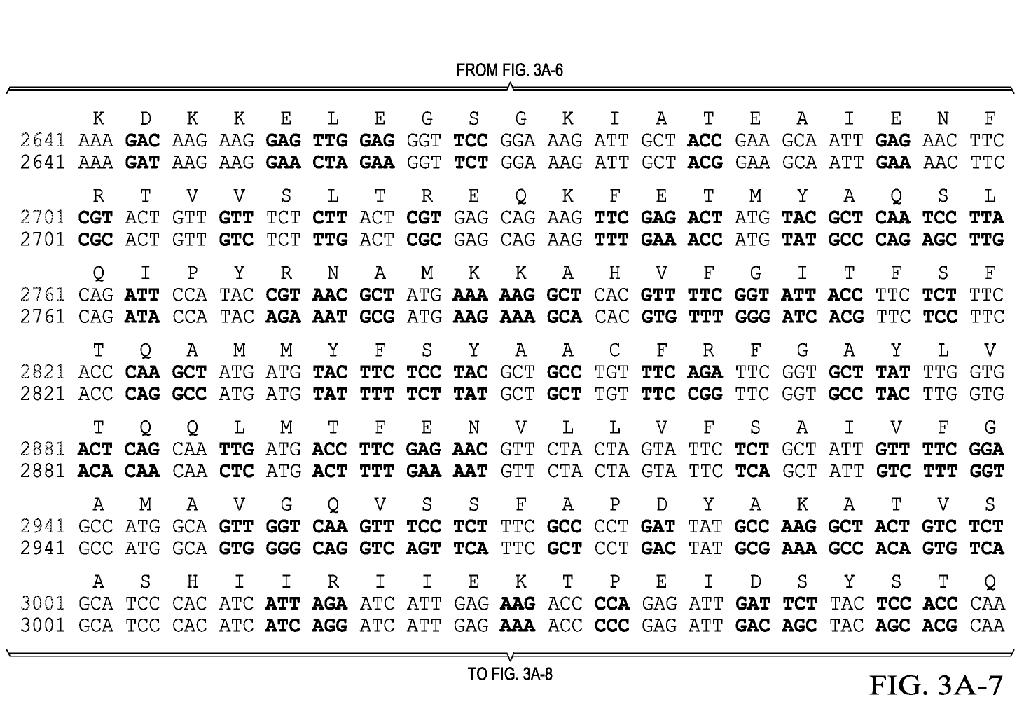

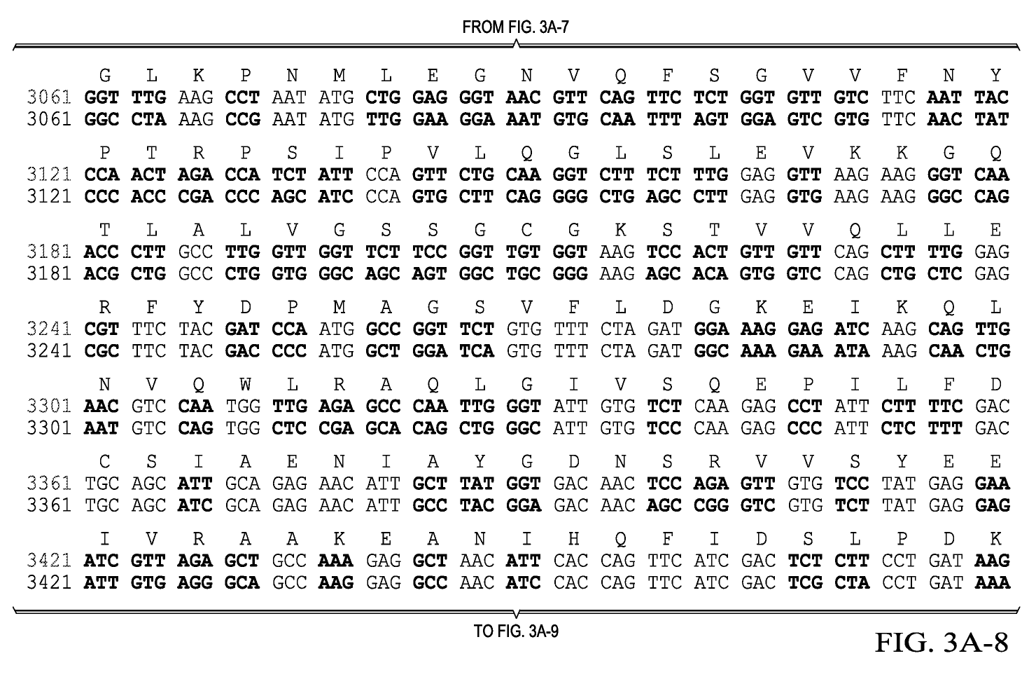

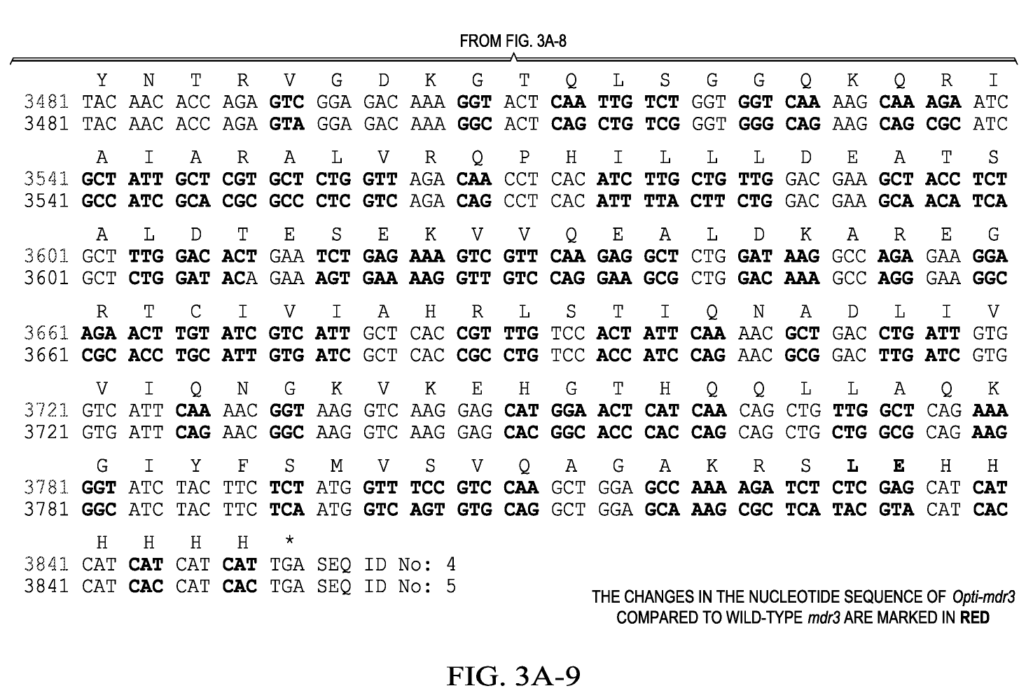

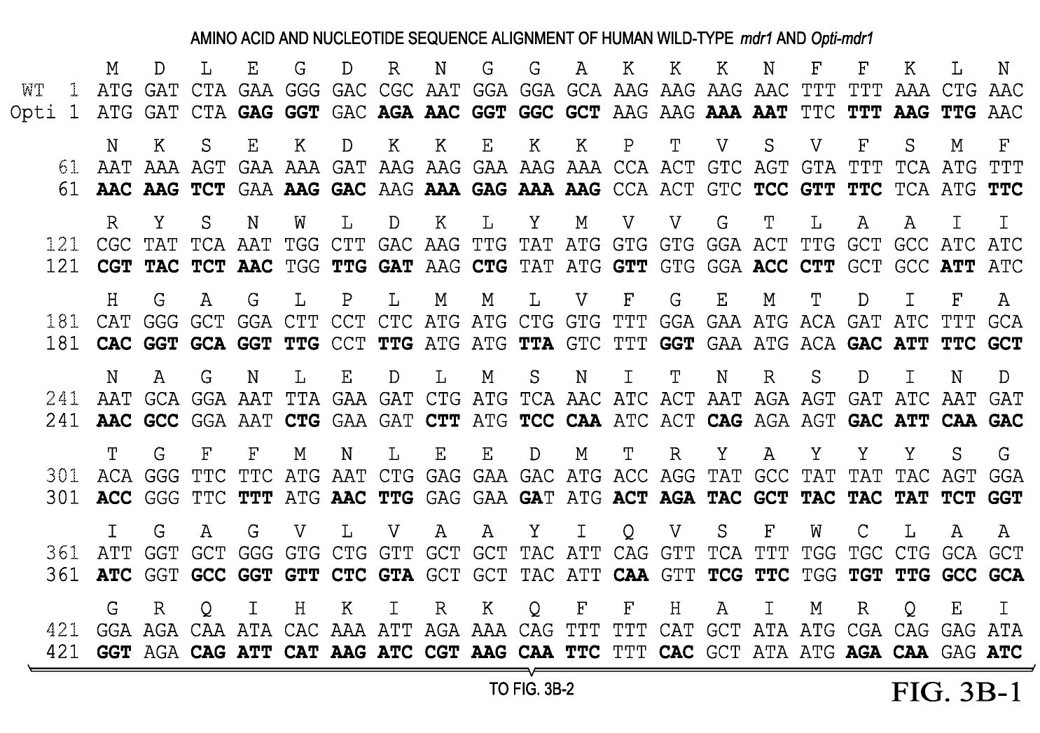

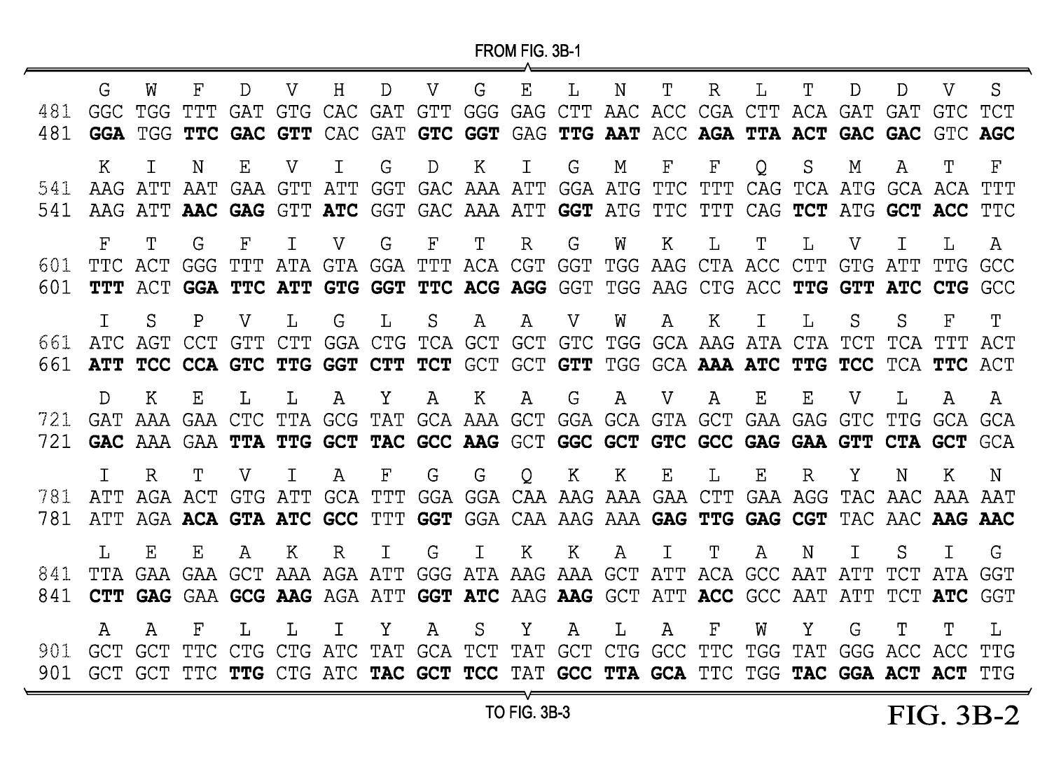

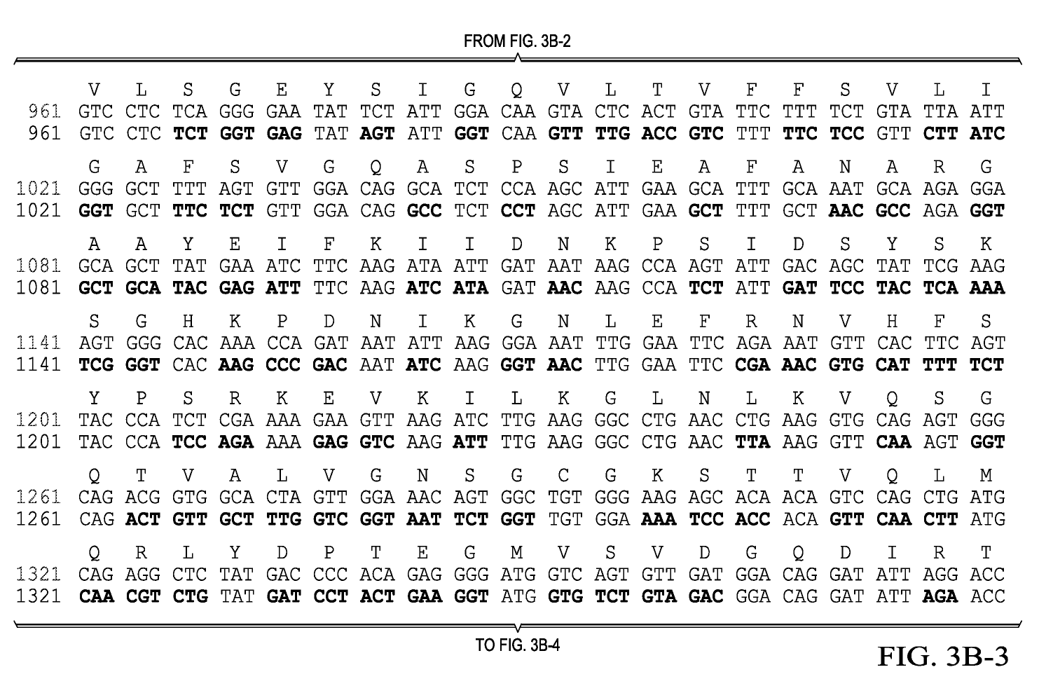

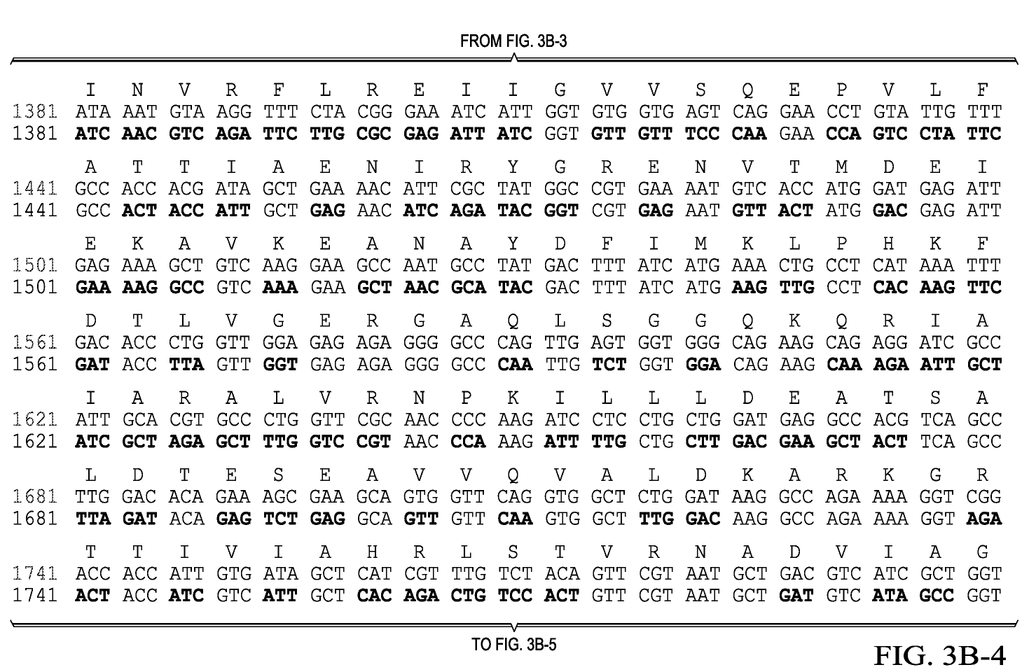

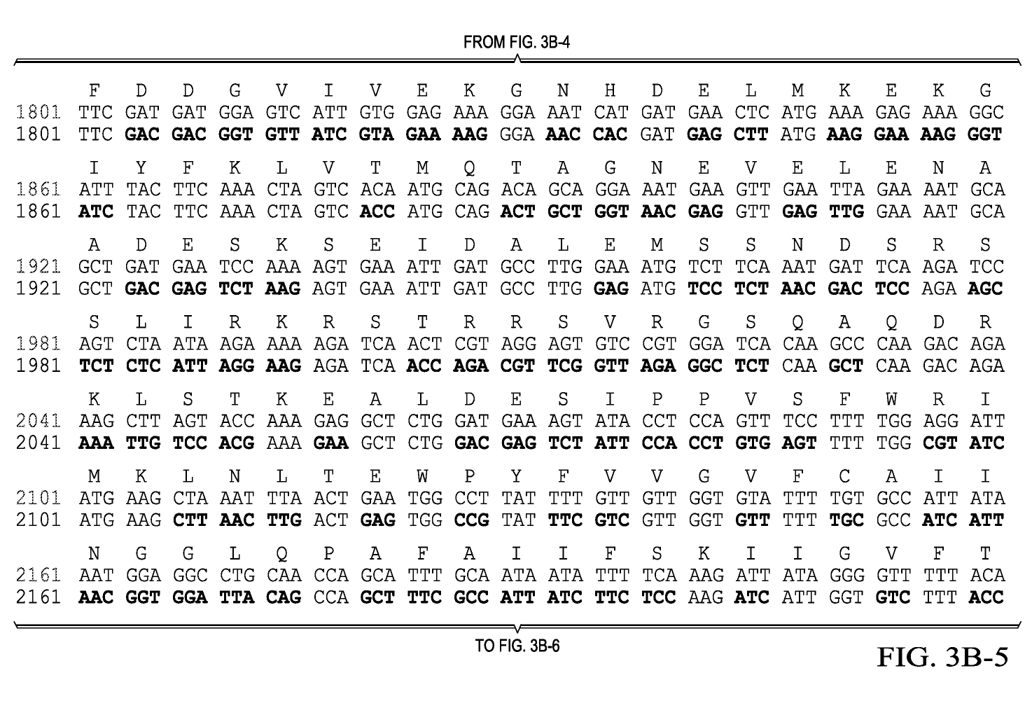

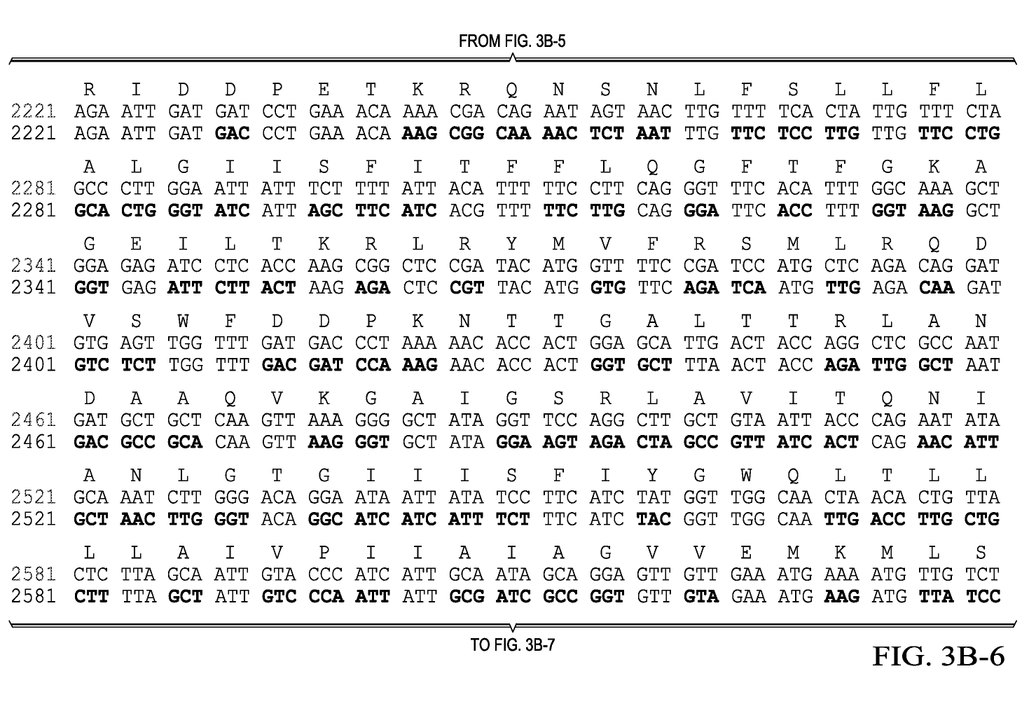

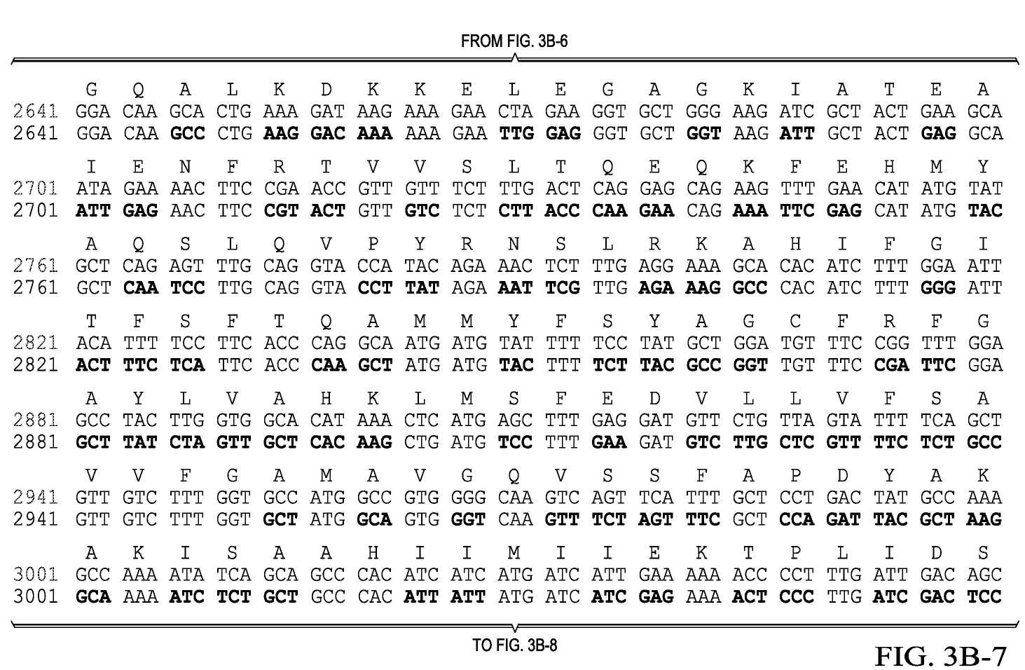

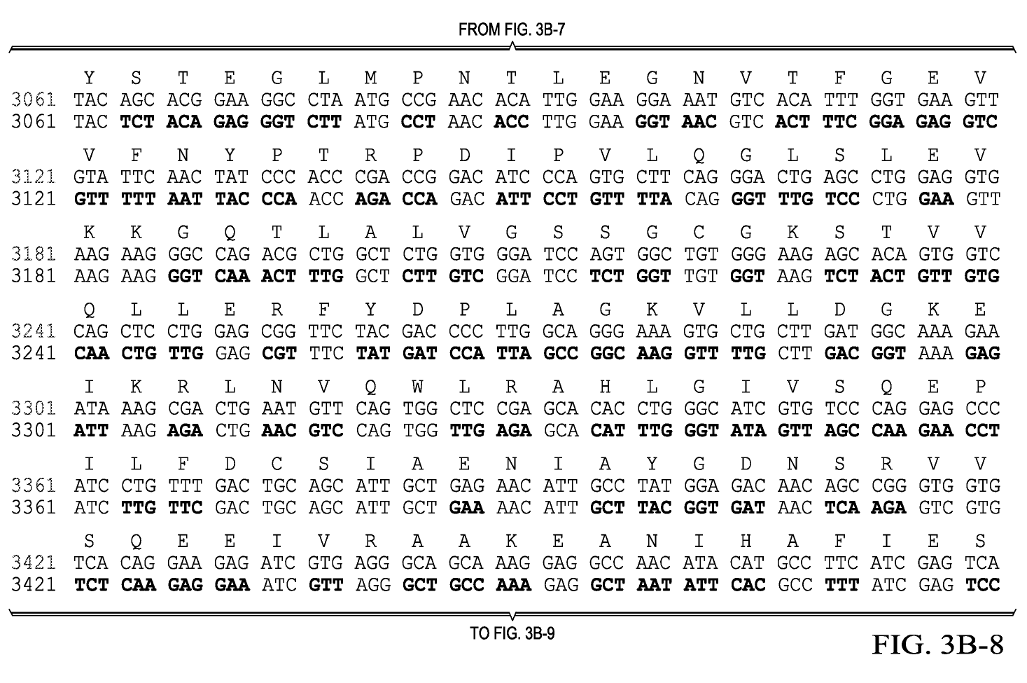

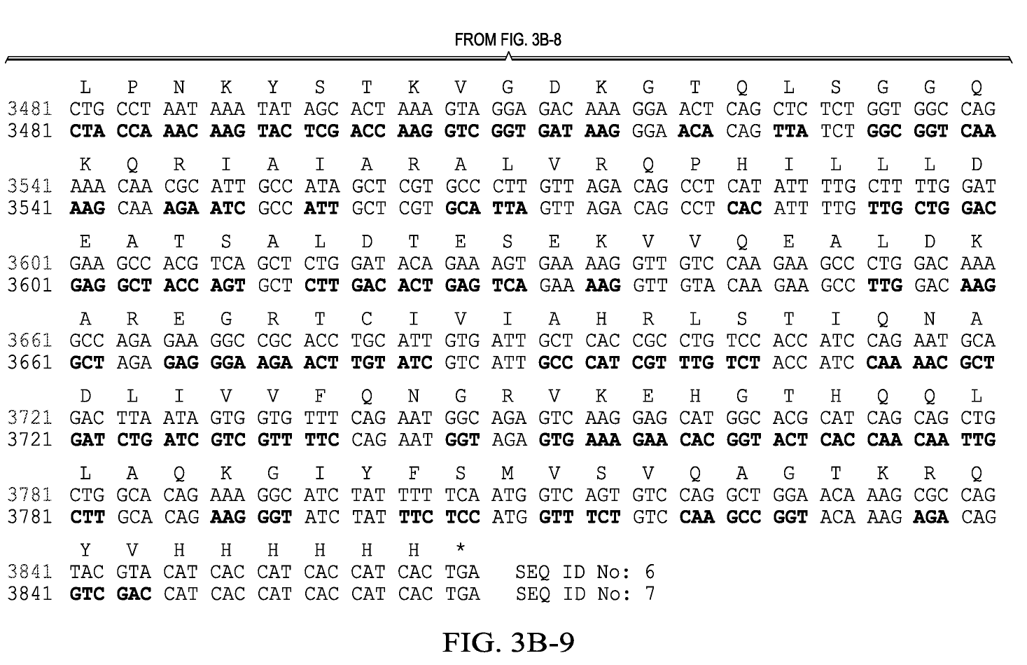

FIG. 3 is an image of the cloning strategy for pLIC-H6 vector and expression in P. pastoris. Schematic representation of the expression construct for ligation-independent cloning (LIC) using the pLICH6 vector described in [4]. Single-stranded overhangs, produced by the 3′ to 5′ exonuclease reactivity of T4 DNA polymerase in the presence of dGTP and dCTP, are shown for the PCR-amplified gene (top) and the corresponding counterparts in the vector (bottom), respectively. After cloning, the pLIC-H₆ plasmid encodes a protein bearing a C-terminal His₆ tag. In addition, the vector contains Kozak-like bases in the region around the ATG start codon (positions −3 and +1) important for high-level expression in P. pastoris [4]. Integrity of the CDS was confirmed by DNA sequencing. The resulting plasmids pLIC-MDR3-H₆ and pLIC-opti-MDR3-H₆ were transformed into P. pastoris strain KM71H and selected on 100 μg/ml Zeocin as described [5].

Cloning of Opti-Pgp and Expression in S. cerevisiae—

The full-length coding sequence of opti-MDR3 was first cloned into the P. pastoris vector pLIC-H₆ via ligation-independent cloning as described in [31], introducing a Kozak-like sequence around the ATG start codon and a His₆-tag at the C-terminus. For direct comparison of gene expression, WT MDR3 was also cloned into pLIC-H₆ using the same strategy (simultaneously removing 5′- and 3′-untranslated regions). The resulting plasmids were named pLIC-opti-MDR3-H₆ and pLIC-MDR3-H₆. Then, opti-MDR3 (including flanking BstBI and Agel restriction sites) was PCR amplified using PfuUltra II and primers SEQ ID No 2 5′-TTCGAAAAAAAAATGGAGTTGG-3′ (forward) and SEQ ID No: 3 5′-ACCGGTTCAATGGTGGTGATGGTGGTGCTCGAGAGATCTTTTGGC-3′ (reverse), then cloned into the PvuII and BamHI sites (blunt-ended with T4-DNA polymerase) of the pVT vector [12, 32] to generate pVT-opti-MDR3. The integrated full-length ORFs from three individual plasmids were confirmed by DNA sequencing. These three plasmids as well as the p VT vector control and the WT gene in pVT (previously named pVT-MDR3.5 [12]), were transformed into S. cerevisiae strain JPY201 (MATaste6Δura3) and selected on uracil-deficient medium as described [12]. 50 to 100 colonies of each transformant were collected into 5 ml of uracil-deficient medium and the mass populations stored at 4° C. for up to two weeks; aliquots were frozen as glycerol stocks at −70° C. Mass populations were grown overnight in uracil deficient medium to an OD₆₀₀ of 1 for protein expression and functional analyses. For Western blot analysis, microsomal membranes were processed from 10 ml cultures [13] and the protein concentrations determined with the Bradford protein assay (BioRad) using BSA as a standard. Equal amounts of membrane protein (15 μg) were resolved on SDS-gels, transferred to a nitrocellulose membrane and stained with Ponceau S (total protein loading control). After washing, the immunoblots were developed with the monoclonal C219 antibody (Covance SIG-38710) and the enhanced chemiluminescence SuperSignal West Pico ECL kit (Pierce). The films from different exposure times were scanned and analyzed using the NIH software package Image J.

Functional Analysis of Opti-Pgp in S. cerevisiae

FK506 resistance and mating assays were as previously described [12] with the following modifications. To measure FK506-resistant growth, overnight cultures were grown in uracil-deficient medium, diluted to an (OD₆₀₀ of 0.05, seeded into sterile 96 well plates in triplicate and grown in YPD medium at 30° C. in the absence or presence of FK506, valinomycin [12, 33], or doxorubicin. OD₆₀₀ was measured at 2 hour intervals for 30 hours in a microplate reader (Benchmark Plus, BioRad) after vigorous mixing. Drugs were dissolved in dimethylsulfoxide and diluted into the plate medium such that the final concentration of solvent was ≦1%. For mating assays, mass populations were diluted to OD₆₀₀ of 0.6, and 0.75 ml were spotted with 0.25 ml of α-type tester strain DC17 (OD₆₀₀ of 1.2) onto a 22 mm 0.45 μm HA filter (Millipore, cat no SAIJ791H5), placed on a YPD plate and incubated for 4 hours, then plated in duplicate on minimal and uracil-deficient medium as described [12, 34]. Mating frequency was calculated as the ratio of transformed cells forming diploid colonies on selective medium to the total number of cells introduced in the assay. Statistical analysis of the functional assays was done with the SigmaPlot 11 software using One Way ANOV A with the pairwise multiple comparison Tukey test.

Expression and Purification of WT- and Opti-Pgp from P. pastoris—

Transformation of P. pastoris strain KM71H and expression analysis were as previously described [31, 35]. Selected strains were grown in a BioFlow IV fermentor and the proteins purified as previously described [13] with the following modifications: 10 mM DTT was included during cell breakage in a glass bead beater to fully reduce the proteins, and all buffers for membrane preparation and chromatography were supplemented with 1 mM β-mercaptoethanol and 0.1 mM tris(2-carboxyethyl)phosphine (TCEP) to keep proteins reduced. Proteins were concentrated to approximately 1 mg/ml using YM-100 Ultrafilters (Millipore). The concentrated protein was aliquoted and stored at −80° C. For gel filtration chromatography, protein was concentrated to 4 mg/ml and 0.5 ml chromatographed on Superose 6B (10×300 mm, GE Healthcare) in 20 mM Hepes-NaOH pH 7.4, 10% glycerol, 50 mM NaCl, 1 mM DTT and 0.2% n-Dodecyl-β-Dmaltopyranoside (DDM) using an Akta Purifier chromatography system (GE Healthcare). Pgp concentrations were routinely determined by UV spectroscopy at 280 nm using a calculated extinction coefficient of 1.28 per mg/ml. Serial dilutions of WT- and Opti-Pgp preparations were further assayed side-by-side with the colorimetric BCA protein assay (Pierce) using BSA with appropriate buffer controls as a standard; the two assays gave essentially the same results. Finally, increasing concentrations of different protein preparations were resolved side-by-side on Coomassie-stained SDS-gels (as in FIG. 2A), individual lanes were scanned and the amount of protein in the Pgp and other protein bands quantitated using ImageJ. The latter method permits visual inspection as well as quantitative validation of samples and allows for direct comparison of the Pgp content of the samples.

ATPase Assays—

Purified Pgp in 0.1% DDM was mixed with 10 mM DTT on ice for 5 min, then activated with 1% E. coli polar lipids for 15 minutes at room temperature followed by 30 s bath sonication as described [13]. ATPase activity was measured at 37° C. in a coupled assay utilizing an ATP-regenerating system [36]. For each well of a 96-well plate, 10 μl (5 μg) of activated wild type (WT) Pgp or Opti-Pgp was added to 200 μl of assay medium containing 10 mM ATP, 12 mM MgSO₄, 3 mM phosphoenolpyruvate, 0.3 mM NADH, 0.5 mg/ml of lactate dehydrogenase, 0.5 mg/ml of pyruvate kinase, 0.1 mM EGTA and 40 mM Tris-HCl, pH 7.4. Verapamil was added from stock solution in water; cyclosporine A was added from concentrated stock in DMSO such that the final DMSO concentration was 2%; control samples contained 2% DMSO. The decrease in NADH absorbance recorded at 340 nm in a microplate reader (Benchmark Plus, BioRad) was linear between 5 and 20 min. ATPase activity was calculated as described previously [37] and plotted with SigmaPlot 10 (Systat Software, Inc.).

Circular Dichroism (CD)—

CD spectra were recorded at 20° C. at a protein concentration of 0.18-0.28 mg/ml in a 0.05 cm cuvette using a thermostated CD spectrophotometer (Olis DSM 1000, USA). Reference and sample buffers contained 5 mM HEPES, pH 7.6, 12 mM NaCl, 2.5% glycerol, 0.05% DDM and 0.25 mM DTT. The rr-helical content was determined by the method of Chen et al., (37).

Scanning Calorimetry (DSC)—

Calorimetry was routinely carried out in 20 mM HEPES, pH 7.6, 50 mM NaCl, 10% glycerol, 0.1% DDM and 5.5 mM DTT in 0.13 mL cells at a heating rate of 2 K/minutes with the VP-Capillary DSC System (MicroCal Inc., GE Healthcare). An external pressure of 2.0 atm was maintained to prevent possible degassing of the solutions on heating. Thermal unfolding was irreversible, as determined by sample cooling and reheating. Heat capacity curves were corrected for instrumental baseline obtained by buffer scans. Separated DSC scans were conducted for buffer containing 1% lipids and no transition was detected in the temperature range of thermal unfolding for the proteins in presence of lipids. DSC data were analyzed with the MicroCal Origin software to obtain the unfolding temperature (Tₘ) and the total unfolding enthalpy (ΔHcal).

Trypsin digestion and SDS-PAGE—

Pgp (5 μg), activated with 1% E. coli lipids, was mixed with 2 μl of trypsin (serially diluted in 1 mM HCl from 1.6 to 0.0001 mg/ml). After 15-minute incubation at room temperature, digestion was stopped with 2 μl (5 ug) of trypsin inhibitor (Type I-P from bovine pancreas, Sigma-Aldrich). Samples were mixed with ≧0.3 volumes of sample buffer (125 mM Tris-C1, pH 6.8, 5% (w/v) SDS, 25% (v/v) glycerol, 0.01% pyronin Y, and 160 mM DTT), incubated for 10 minutes at RT, then resolved on 10.5-14% polyacrylamide gradient Criterion precast gels (BioRad), and stained with Coomassie Blue.

Codon Usage Bias in P. pastoris—

A codon usage table (seen in FIGS. 1A-1C) for 30 native genes known to be expressed at high levels in P. pastoris was prepared [29, 30, 38, 39]. Although the table was based on a modest number of genes, the resulting codon usage frequencies were quite comparable to those of 263 highly expressed genes in the related yeast S. cerevisiae [15]. For example, the most abandoned codon for each amino acid as well as the codons used at low frequency (<1 0%, highlighted in orange) were very similar in both species of yeasts (compare columns 3 and 4, FIGS. 1A-1C). However, codon frequencies were distinctly different from those in the Kazusa or the Pichia genome databases, which do not discriminate between poorly and highly expressed genes. Besides five low frequency (<10%) codons seen in the Kazusa database, an additional 18 codons occur only at low frequency among highly expressed genes (compare columns 1 and 2 versus 4, FIGS. 1A-1C). Thus, codon usage was considerably more stringent for high level compared to low or medium level expression. Also, among highly expressed genes certain high frequency codon preferences were inverted: CAC over CAU (73:27%) for His, UUC over UUU (67:33%) for Phe, GAC over GAU (59:41%) for Asp and GAG over GAA (58:42%) for Glu. Consequently, adoption of codon frequencies seen in highly expressed genes may represent a better choice for optimization of genes for high level expression.

Optimization of the Pgp Gene—

Codon frequencies within the 3828 bp coding sequence of the native mouse MDR3 gene (also called MDRla or abcbla) differed markedly from those of P. pastoris highly expressed genes, with pronounced over-representation of yeast low frequency codons and under-representation of yeast preferred and higher frequency codons (see column 5, FIG. 1A-1C). In addition, the native gene sequence showed 38 tandem codon repeats, 99 regions of extended secondary mRNA structure (hairpin loops) that can hinder translation, 86 AT-rich or GC-rich regions (up to 10 bases in length), 9 cryptic splice sites, and a GC content of 48% which is somewhat higher than that found in highly expressed Pichia genes (45%). These structural elements, along with the codon bias, appeared unfavorable for high-level expression in P. pastoris, and our strategy to optimize the MDR3 sequence was as follows: We omitted all occurrences of the 19 low frequency codons (<8%) and we set the relative frequencies among the remaining codons similar to those of highly expressed genes. We also avoided codon repeats and AT-rich regions, and adjusted the GC content to 45% (balanced to ±10% within a 40 bp window throughout the gene) (FIG. 2B).

FIG. 3A is an amino acid and nucleotide sequence alignment of wild-type MDR3 and Opti-MDR3. FIG. 3B is an amino acid and nucleotide sequence alignment of human wild-type MDR1 and Opti-MDR1. The resulting gene sequence (“opti-MDR3”) is given in FIG. 3 (GenBank JF834158) and the final codon usage is shown in FIGS. 1A-1C, column 6. The changes in the nucleotide sequence of Opti-MDR3 compared to wild-type MDR3 and wild-type MDR1 and Opti-MDR1 are marked in red.

Functional Analysis of Opti-Pgp in S. cerevisiae—

Because codon usage of highly expressed genes is so similar in S. cerevisiae and P. pastoris, we expected our optimization approach to improve expression in both yeasts. For three mass populations of independent S. cerevisiae transformations, Pgp-specific signal intensities in Western blots of microsomal membranes indicated that Opti-Pgp transformants expressed the protein at two- to three-fold higher levels than did WT-Pgp transformants (FIG. 1A). This indicated that gene optimization indeed enhanced expression levels in yeast.

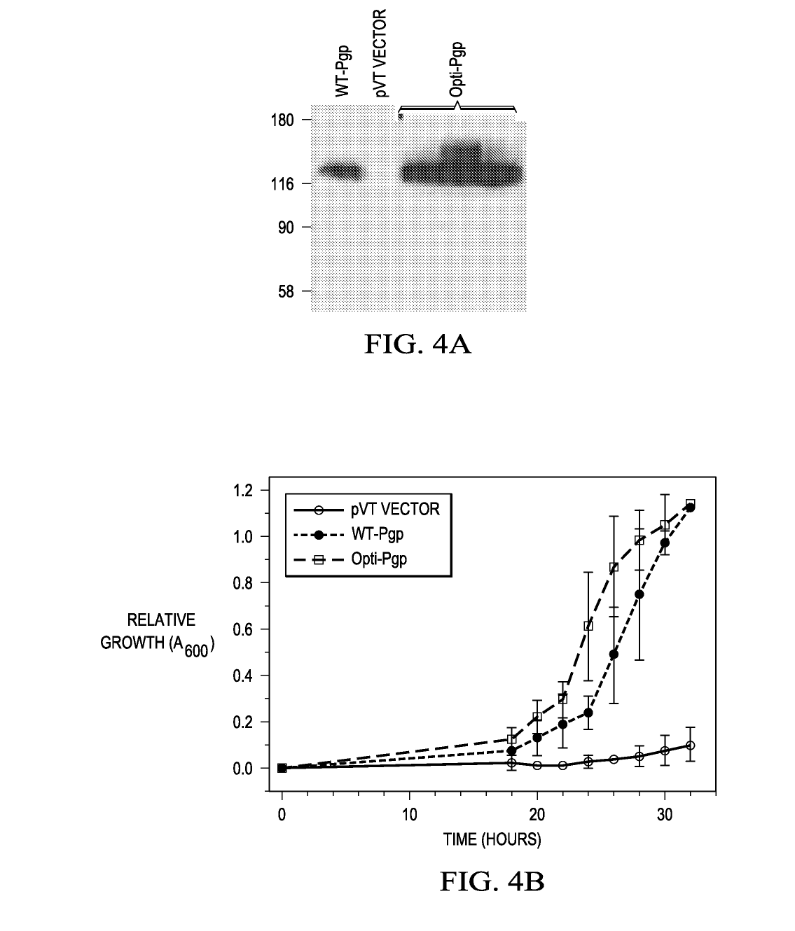

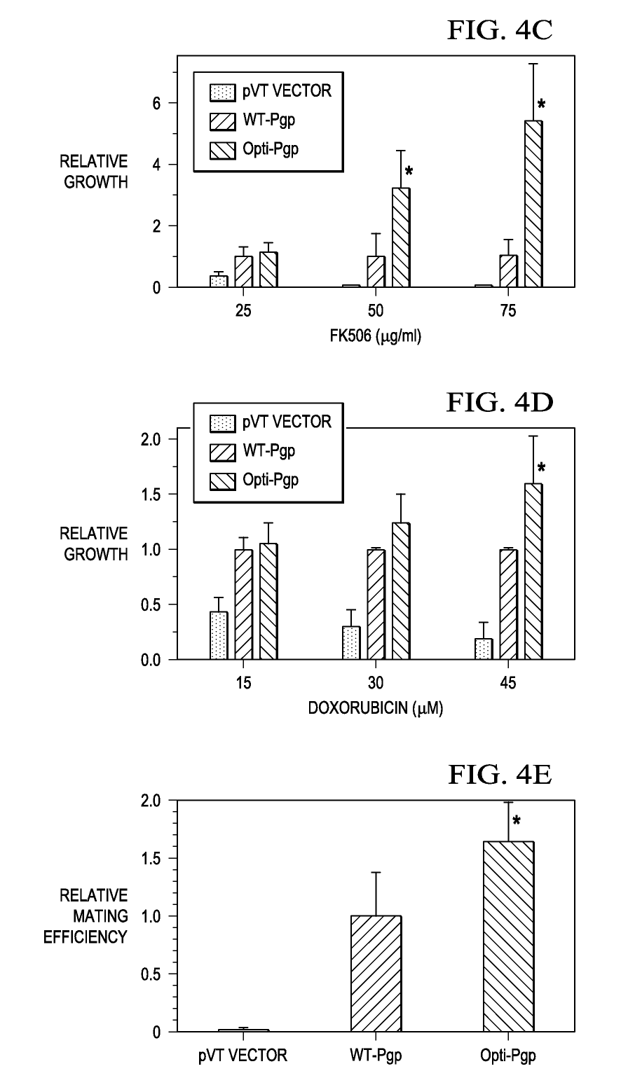

FIGS. 4A-4E are images the protein expression levels and in vivo biological activity of WT- and Opti-Pgp in S. cerevisiae. FIG. 4A is an image of three independent pVT-opti-MDR3 clones were transformed into S. cerevisiae, microsomal membrane proteins (15 μg) of mass populations resolved on a 10% SDS-gel and the Western blot probed with the Pgp-specific monoclonal C219 antibody (Covance SIG-38710). Mass populations transformed with p VT vector alone or the WT gene served as controls. The positions of the MW protein markers are indicated in kDa.

FIG. 4B is an image of a graph showing the growth resistance to the fungicide FK506 (50 μg/ml) was monitored at A₆₀₀ for wild-type Pgp (WT-Pgp), gene-optimized Pgp (Opti-Pgp) and control pVT vector transformants. Data points represent the mean±standard deviations of three independent transformants assayed in triplicate in four independent experiments; where not visible, error bars are smaller than the plot symbol. FIG. 4C is an image of a graph showing the growth of individual mass populations in the absence or presence of increasing concentrations of FK506 (25, 50 and 75 μg/ml) was measured at A₆₀₀ after 25-26 hours and is expressed as growth relative to WT-Pgp.

FIG. 4D is an image of a graph showing the growth resistance in the absence or presence of doxorubicin (15, 30 and 45 μM) was measured relative to WT-Pgp. FIG. 4E is an image of a graph showing the mating frequency represents the proportion of transformed a-type JPY201 cells that formed diploids upon mating with R-type tester cells DC17, followed by plating on minimal medium [34]. Values are expressed as a percentage of the WT frequency±the standard deviation of four experiments using three independent transformants. Asterisks indicate significant differences between WT- and Opti-Pgp (p<0.05).

Although the optimized gene encodes identical primary amino acid sequence to the WT protein, co-translational effects might cause changes in protein folding [40]. Therefore, it was important to demonstrate that Opti-Pgp retained full biological activity. Procedures to test in vivo Pgp function in P. pastoris have not been developed, so to take advantage of established biological assays [12, 33, 34] and to examine substrate specificity, we first tested Opti-Pgp function in the yeast S. cerevisiae. We previously showed that expression of native Pgp in S. cerevisiae confers drug resistance against fungicides [12, 33, 41], so we first measured growth resistance of mass populations to the macrolide immunosuppressant FK506. In four independent experiments Opti-Pgp transformants grew faster than WT-Pgp in the presence of FK506, i.e. they entered log-phase growth approximately 22 hours after inoculation and reached stationary phase at approximately 28 hours, two hours sooner than WT-Pgp (FIG. 4B). Similarly, growth of OptiPgp transformants in the presence of the cyclic peptide ionophore valinomycin (80 μg/ml) appeared to be as good as or better than WT-Pgp transformants (data not shown). To better assess potential differences in growth resistance between WT- and Opti-Pgp transformants we grew the cultures in the presence of increasing concentrations of FK506 (FIG. 4C). At concentrations of 25 μg/ml FK506 no difference was evident (pairwise Tukey test comparison p=0.577) but at the higher concentrations of 50 or 75 μg/ml FK506 Opti-Pgp cultures grew significantly faster than Wt-Pgp (p=0.025 and 0.003, respectively). Pgp is known to convey multidrug resistance by transporting a wide variety of structurally unrelated compounds. To demonstrate that polyspecificity was maintained in the Opti-Pgp we also measured its ability to confer S. cerevisiae with resistance to the anticancer drug doxorubicin. At concentrations of 15 and 30 μM doxorubicin, a pairwise comparison (Tukey test) between WT- and Opti-Pgp revealed no significant difference (p=0.809 and 0.197) but at the higher concentrations of 45 μM doxorubicin Opti-Pgp cultures grew significantly faster than WT-Pgp (p=0.034, FIG. 4D). The data demonstrate that Opti-Pgp, like WT-Pgp, transported a range of fungicidal and anticancer drugs. Higher protein expression levels in the Opti-Pgp strains (FIG. 4A) likely accounted for their enhanced drug resistance compared to the WT-Pgp strains.

Pgp also imparts S. cerevisiae with the capacity to export a-factor mating peptide, permitting diploid formation that can be efficiently measured in a mating assay [12, 33]. Thus we also compared the capacity of Opti-Pgp to restore mating in the sterile ste6Δ yeast strain JPY201. Mating frequencies of Opti-Pgp transformants were about 1.5-fold higher than WT-Pgp controls (p=0.021, FIG. 4E) indicating that Opti-Pgp can export this pheromone more efficiently than WT-Pgp. Together, the results of functionality studies were consistent with higher protein expression, more effective folding and/or more complete trafficking of Opti-Pgp to the cell surface where it executes its biological activity.

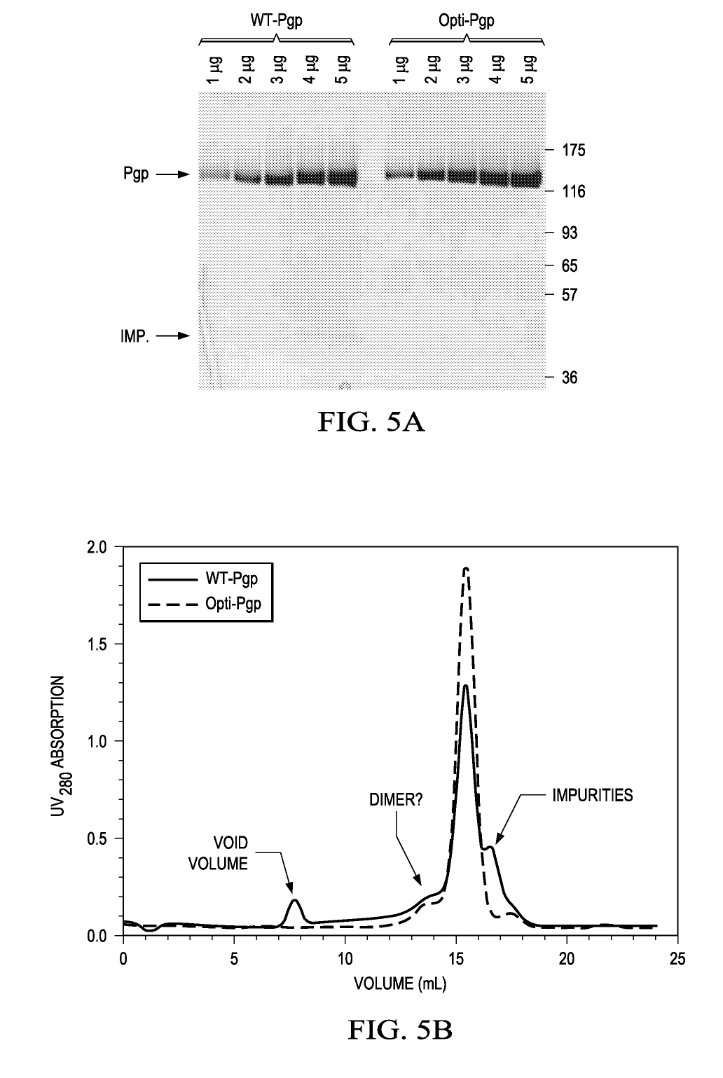

FIGS. 5A and 5B are images of the purification and size exclusion chromatography of WT- and Opti-Pgp from P. pastoris. FIG. 5A is an image of proteins purified from P. pastoris fermentor cultures by chromatography on Ni-NTA and De52 resin. Increasing amounts of proteins (1 to 5 μg) were resolved on a 10% SDS-gel and stained with Coomassie Blue. The positions of the MW protein markers are indicated in kDa; the protein band labeled “Imp.” (impurities) did not cross-react with the Pgp specific antibody C219. FIG. 5B is an image of two milligrams (500 μl) of purified, detergent soluble proteins were loaded on a Superose 6B column and resolved in buffers containing small amounts of detergent (see Materials and Methods). A representative of four independent runs is shown for WT-Pgp (solid line) and Opti Pgp (dotted line). Molecular mass markers were resolved under identical buffer conditions, the elution volumes were as follows: Blue-dextran (void volume) 6.7 ml, thyroglobulin (669 kDa) 12.4 ml, ferritin (440 kDa) 14.2 ml. aldolase (158 kDa) 15.8 ml, conalbumin (75 kDa) 16.8 ml and ovalbumin (43 kDa) 17.1 ml. The calculated molecular mass of monomeric Pgp (including the His6-tag) is 142 kDa, the predicted detergent micelle size for DDM is about 70 kDa.

Purification of Opti-Pgp from P. pastoris—For large-scale protein production, fermentor cultures of WT- and Opti-Pgp expressing strains of P. pastoris were grown and the proteins purified as described in Materials and Methods [13]. Consistently higher yields of purified proteins were obtained from the Opti-Pgp strain (13±3.2 mg per 100 g cells, n=6) than WT-Pgp (4.3±1.6 mg per 100 g cells, n=3) (Table 1).

TABLE 1 is a comparision of WT-and Opti-Pgp.

| | WT-Pgp | Opti-Pgp |
|---|---|---|
| Yield per 100 g cells | 4.3 ± 1.6 mg | 13.0 ± 3.2 mg |
| Maximal ATPase activity | 1.8 ± 0.24 | 2.1 ± 0.28 |
| (μmol min⁻¹ mg⁻¹) ¹⁾ | | |
| Half-maximal stimulation | 9.1 ± 4.6 | 4.2 ± 2.2 |
| by Verapamil (μM) ²⁾ | | |
| Half-maximal inhibition | 0.98 ± 0.24 | 1.1 ± 0.26 |
| by cyclosporine A (μM) ²⁾ | | |

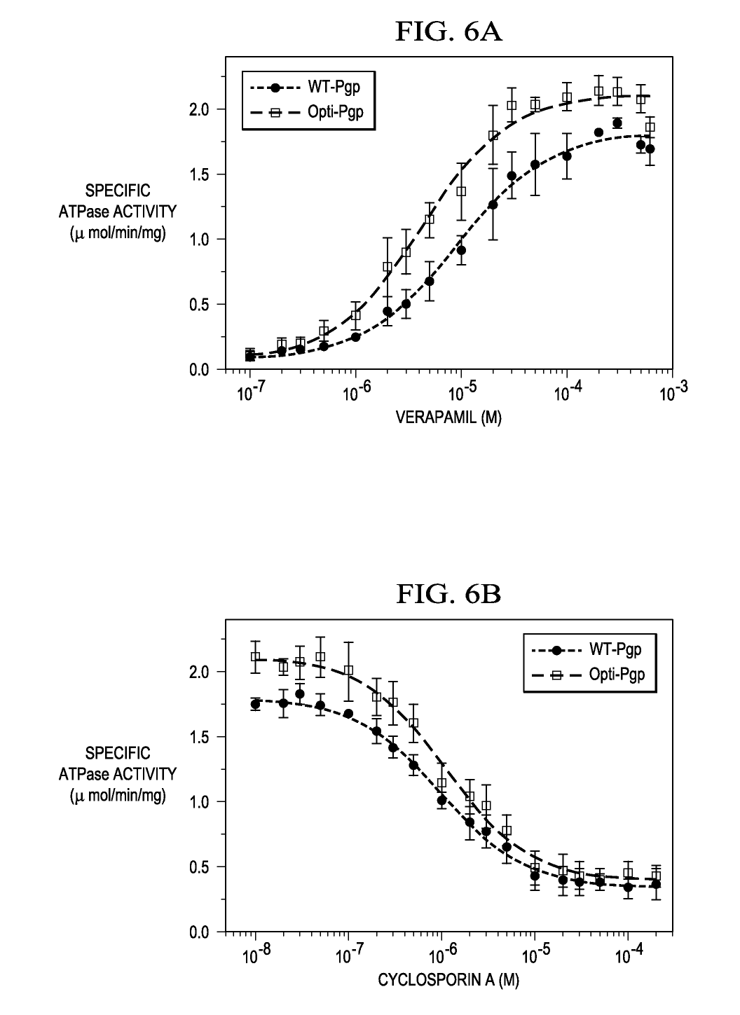

¹⁾ Average and standard deviations (n > 30) from at least three independently purified preparations.
²⁾ Concentrations required for half-maximal stimulation or half-maximal inhibition of ATPase activity were calculated from the fits shown in FIGS. 5 and 6, respectively. Standard deviations are given for individual fits from three independent experiments.

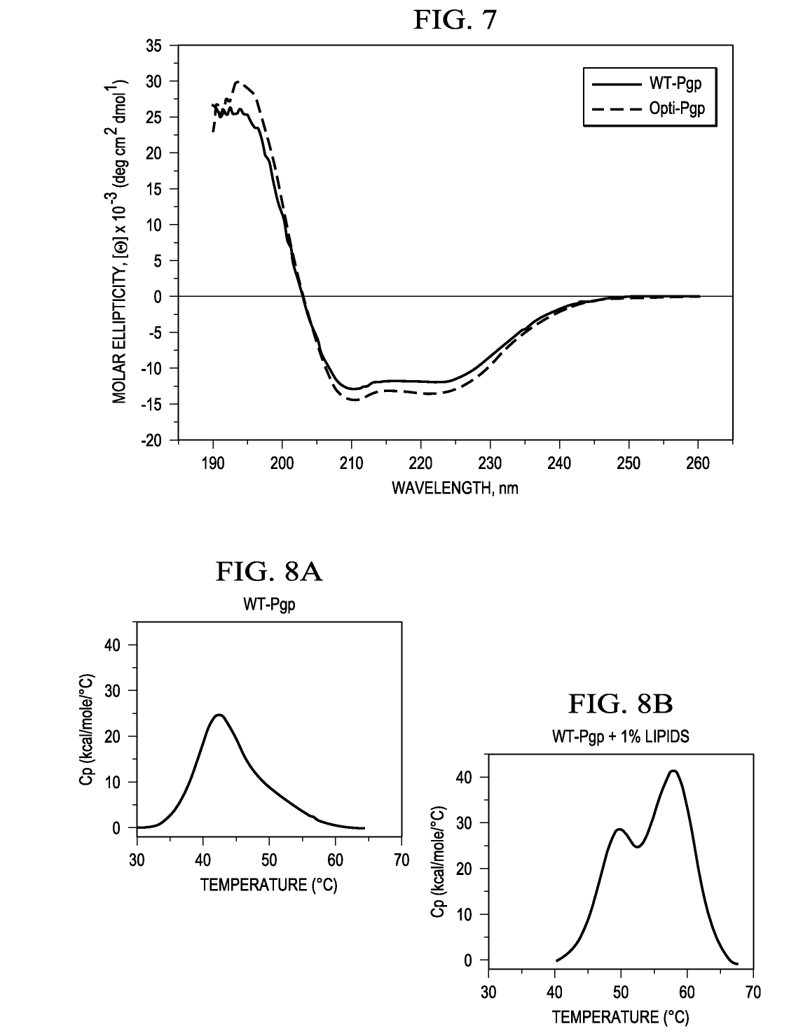

Perhaps as a result of yield, purified Opti-Pgp preparations also exhibited lower residual contaminant levels than the 5-10% seen in WT-Pgp preparations on Coomassie-stained gels (labeled “imp.” in FIGS. 5A and 7) and on size exclusion chromatography (SEC) (FIG. 5B). WT-Pgp preparations showed a peak at the void volume of the column (FIG. 5B, solid line) that was not seen with Opti-Pgp (dotted line) suggesting that the latter protein is less prone to aggregation. In both cases the major protein peak appeared monomeric with an elution volume (15.3 mL) indicating an apparent size of approximately 200 kDa, and a minor peak at 13.5 mL consistent with Pgp oligomer [42]. Thus, gene-optimization improved the quality of the purified protein, as collectively evidenced by the higher yield and purity of Opti-Pgp preparations, its monodispersity, and its resistance to aggregation.

FIGS. 6A and 6B are images of graphs of stimulation and inhibition of ATPase activity. FIG. 6A is an image of a graph of stimulation and inhibition of ATPase activity. The ATPase activity of purified WT- and Opti-Pgp was assayed in the presence of increasing concentrations of verapamil. The solid lines are non-linear regression fits to the equation f=d+(a*x^{b}/(c^{b}+x^{b})), where d is the activity in the absence of verapamil (basal activity), a is the maximum verapamil-stimulated activity, b is the Hill coefficient, c is the concentration for half-maximal stimulation, and x is the concentration of verapamil. No cooperativity was observed with Hill coefficients close to 1.0 (0.998 and 1.05, respectively). Each data point represents the mean from at least 3 independent experiments (from three different protein purifications)±standard deviation. FIG. 6B is an image of a graph of the purified proteins were assayed in the presence of 150 μM verapamil to maximally stimulate ATPase activity but with increasing concentrations of the inhibitor cyclosporine A. The solid lines are non-linear regression fits to the equation f=a−(e*y^{b})/(c^{b}+y^{b})), where e is the maximum inhibition, and y is the concentration of cyclosporine A. No cooperativity was observed with Hill coefficients close to 1.0 (0.95 and 0.98, respectively).

ATPase activity of purified Opti-Pgp-ATPase activity of Opti-Pgp in the presence of 150 μM verapamil was 2.1±0.28 μmol/min/mg (n>30) and was somewhat higher than WT-Pgp (1.8±0.24 μmol/min/mg, n>30), consistent with the low-level impurities and aggregation products present in WT-Pgp preparations (FIGS. 5A and 5B). The half-maximal stimulatory concentrations for verapamil were 4.2 and 9.1 μM for Opti- and WT-Pgp, respectively (FIG. 6A), not significantly different in the two tail test (p=0.24). Inhibition of the verapamil-stimulated ATPase activity by the immunosuppressant cyclosporine A was also comparable for the two proteins, with half-maximal inhibition seen at 0.98 μM and 1.1 μM for Opti- and WT-Pgp, respectively (p=0.588, FIG. 3B). The enzymatic data indicate unaltered affinities for substrates and inhibitors in the purified proteins.

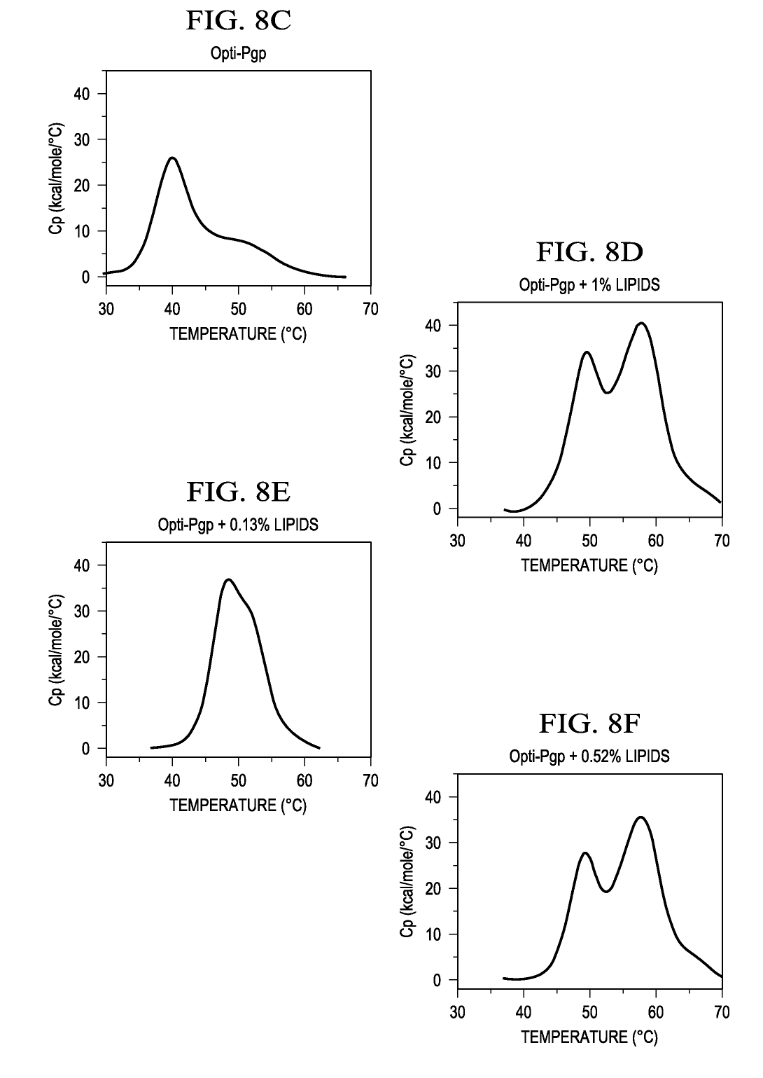

FIG. 7 is an image of the CD spectra of WT- and Opti-Pgp. CD spectra of the purified proteins were recorded after buffer exchange by size-exclusion chromatography (peak fractions from FIG. 8B). Protein concentrations were determined by UV spectroscopy, as well as the colorimetric BCA protein assay using BSA as a standard; the two assays gave essentially the same results. Each spectrum represents an average of 10 scan from three different protein preparations. Molar ellipticity values were calculated according to [Θ]=Θ (100×MRW/lc), where Θ is the measured ellipticity in degrees, MRW is the molecular weight of Pgp (141,000 g/mol), 1 is the path length in centimeters, and c is the concentration of the protein in grams per liter [43].

CD Spectroscopy—

To monitor potential differences in secondary structure, WT- and OptiPgp were investigated by far-UV CD (FIG. 7). The shape of the curves was essentially identical, as was the size of the peak near 220 nm, suggesting the presence of a significant amount of α-helicity. In fact, the α-helical content was estimated to be approximately 41% for WT- and 46% for Opti-Pgp using the method of Chen et al. [43]. These values are very close considering that accurate protein concentration determination is critical for these estimates.

FIGS. 8A-8F are images of the Differential Scanning calorimetry of WT- and Opti-Pgp. Purified proteins were exchanged into buffer containing a defined DDM concentration (as in FIG. 5B), and the temperature dependence of the molar heat capacity recorded; protein concentrations ranged between 0.45-0.78 mg/ml for WT-Pgp and 0.58-0.78 mg/ml for Opti-Pgp, respectively. FIGS. 8A and 8C: no lipid added. FIGS. 8B and 8D: Proteins were preincubated with 1% (w/w) E. coli lipid (lipid to protein ratio of 16:1, w/w) for 15 min at RT followed by 30 s bath sonication as described [13]. FIGS. 8E and 8F: Opti-Pgp was preincubated with 0.13% or 0.52% (w/w) E. coli lipid (lipid to protein ratios of 2.2:1 and 8.4:1, w/w)). Control samples containing the same amount of lipid had no detectable transition in the temperature range of protein unfolding.

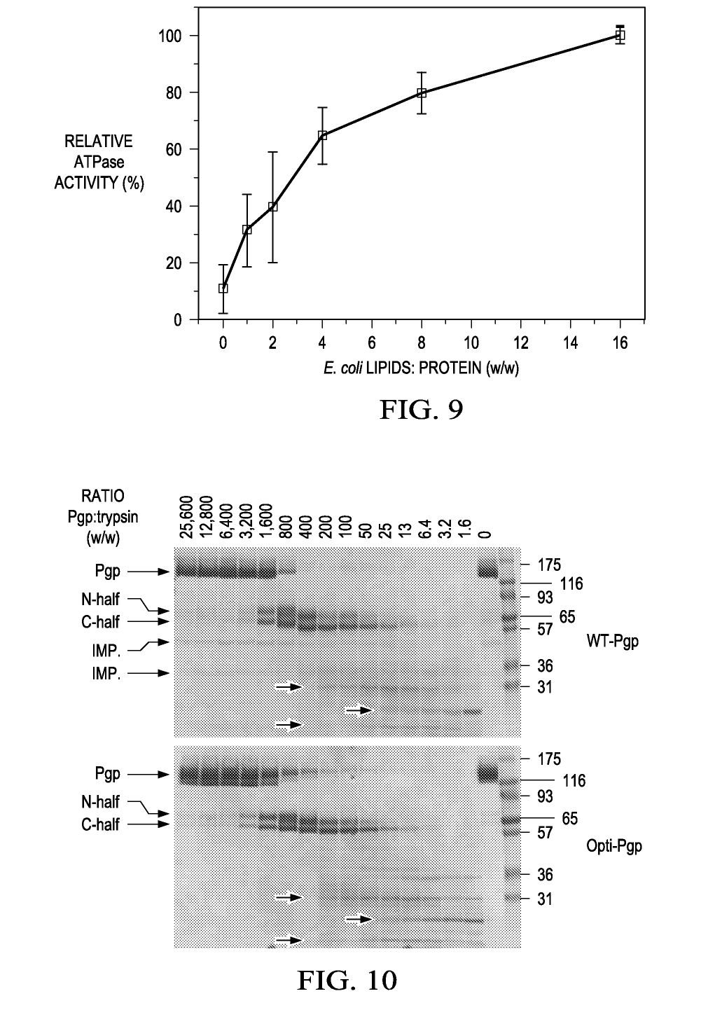

Thermal Unfolding of WT- and Opti-Pgp—Thermal unfolding was monitored by DSC to directly probe protein stability and cooperativity of unfolding. At the least, a detectable DSC transition supports the presence of a folded, cooperative tertiary structure. Comparison of the upper and middle panels of FIG. 8A-8F shows that the unfolding Tₘ and the shape of the unfolding transitions are essentially the same for WT- and Opti-Pgp, whether in detergent solution (FIGS. 8A and 8C) or after addition of 1% lipids (FIGS. 8B and 8D), i.e. under conditions giving maximum ATP hydrolysis rates [13]. The presence of lipid shifted the Tₘ from ˜40° C. (with a minor transition apparent at ˜50° C.) to higher temperatures, with the concurrent appearance of two clear transition maxima near 50° C. and 58° C. (Table 1). The significant increase in the total unfolding enthalpy ΔH_{cal} for both proteins upon lipid addition indicated improved stability and suggested an increase in stable tertiary structure of Pgp when surrounded by lipids. Further measurements of the thermal unfolding of Opti-Pgp at limiting lipid concentrations (FIGS. 8E and 8F) demonstrated that the Tₘ and ΔH_{cal} increased gradually, with a single but asymmetric peak seen at 0.13% lipid while the second transition appeared at lipid concentrations of ≧0.52%. Similarly, verapamil-stimulated ATPase activity of Opti-Pgp showed an increase from 11% in the absence of lipids to 40% and 80% in the presence of 0.13% and 0.52% lipid (FIG. 9).

FIG. 9 is an image of a graph of the lipid dependence of ATPase activity. ATP hydrolysis of Opti-Pgp was assayed after activation with increasing concentrations of E. coli lipids as described in Materials and Methods. Averages±range of two independent experiments are given. 1% lipids added correspond to a lipid:protein ratio of 16:1.

The observation of two defined transitions in the presence of lipid is consistent with the presence of at least two structural domains of different stabilities which, in the absence of lipid, may be energetically equivalent or may not manifest as distinct domains. These are only two possible others may be equally feasible. Taken together, the thermal unfolding profiles are consistent with a folded protein that gains stability and, most likely, structure as a function of lipid concentration.

FIG. 10 is an image illustrating determining the sensitivity of WT- and Opti-Pgp to trypsin. Five μg of purified lipid-activated proteins were incubated with increasing concentrations of trypsin. Samples were resolved on 10.5-14% gradient gels and stained with Coomassie-Blue. The positions of the MW protein markers are indicated in kDa. Arrows indicate the position of the full-length proteins (Pgp), the N-terminal or C-terminal half size proteins, and the position of major tryptic fragments; Imp., impurities.

Tryptic digestion profiles of purified WT- and Opti-Pgp to disclose subtle differences in folding between WT- and Opti-Pgp, we compared their relative susceptibilities to limited proteolysis by trypsin. FIG. 10 shows the disappearance of the Pgp band as a function of trypsin; the concentration required for 50% degradation (expressed here as the ratio of Pgp:trypsin) was the same for WT- and Opti-Pgp. Coincident appearance of the N- and C-terminal half fragments produced by the action of trypsin at the first cleavage sites in the linker region [44] as well as of smaller fragments (36 kDa, 31 kDa and smaller, arrows) at a given concentration of trypsin argues that the principle cleavage sites were equally accessible in the two proteins. This result implied that the two had similar tertiary structures, which was completely consistent with the CD and DSC results.

As a eukaryotic expression system, P. pastoris has many advantages, such as efficient protein folding, membrane targeting, proteolytic processing, disulfide formation and glycosylation [45]. It is a cost-effective system that provides high biomass in fermentor cultures and thus greater amounts of protein per culture volume than any other system, and therefore proved an ideal choice for Pgp production for X-ray crystallography and functional studies [11, 12, 37, 46, 47, 48, 49, 50]. Still, as for any membrane protein, production of pure protein for biophysical and enzymological study is a relentless challenge and any improvements in yield, quality and stability of the protein will greatly facilitate downstream analysis.

To maximize protein expression at the translational level we optimized codon usage in the Pgp gene (mouse MDR3) according to codon frequency found among highly expressed P. pastoris genes, and we also removed mRNA instability motifs and secondary structure that may impair translation [51]. The main purpose of this study was to rigorously analyze the function of gene optimized “Opti-Pgp” in vivo and at the purified protein level to detect any potential differences in function or solution structure, if any, compared to WT-Pgp. Opti-Pgp was expressed at two- to three-fold higher levels and was fully able to convey in vivo drug resistance against a broad range of anticancer drugs and fungicides in the related S. cerevisiae yeast (FIG. 1). Indeed the growth resistance profiles together with the enhanced capacity of Opti-Pgp to export a-factor mating peptide suggested that cotranslational folding and/or trafficking to the cell surface was improved compared to WT-Pgp. Gene-optimization increased Pgp protein production from P. pastoris by about three-fold. ATP hydrolysis by the purified protein was strongly stimulated by verapamil (˜15-fold) and inhibited by cyclosporine A with binding affinities indistinguishable from WT-Pgp (FIG. 6, Table 1). Moreover, ATP hydrolysis rates were enhanced (˜1.2-fold) likely due to the higher purity and/or stability of Opti-Pgp preparations. SEC of Opti-Pgp samples that were frozen and thawed once showed a symmetrical peak with a retention volume corresponding to monomeric protein, and no aggregated protein was detected at the void volume of the column in contrast to WT-Pgp samples (FIG. 5). The functionality data, together with the higher yield and purity, as well as its monodispersity in SEC and lower background protein aggregates in crystallization trays (not shown) suggest that Opti-Pgp will be a most valuable tool for future biophysical studies requiring large amounts of high quality protein.

These important findings were extended further by analyzing purified Pgp conformation by CD, DSC and limited proteolysis. WT- and Opti-Pgp showed very similar CD profiles suggesting an α-helical content of about 41-46% in DDM solution [43], a value somewhat lower than the ˜60% α-helical content calculated from X-ray structures solved in the same detergent [11]. Higher flexibility of the protein in solution and/or the absence of cholate, transport substrate, nucleotide, inhibitors or additives necessary for crystallization may account for this lower helicity value [52, 53, 54]. We previously demonstrated a strong dependence of Pgp ATPase activity on the presence of lipid [13], indicating that lipids promote an active conformation of Pgp, possibly through interactions with the hydrophobic TMDs. Here we show for the first time that the presence of 1% E. coli lipid increased the thermal stability of the protein as indicated by a shift in Tₘ from ˜40° C. to 49° C., as well as a significant increase in the total unfolding enthalpy ΔH_{cal} of both WT- and Opti-Pgp (FIG. 8, Table 2). Table 2 is a table of the thermal unfolding parameters of WT- and Opti-Pgp.

| | Added | Unfolding temperature (° C.) | | ΔH_{cal} | |
|---|---|---|---|---|---|
| Sample | lipids | T₁ ^{a} | T₂ ^{a} | (kcal/mol) | n ^{b} |
| WT-Pgp | None | 43.0 ± 1.6 | ND | 264 ± 87 | 5 |
| | 1% lipid | 50.4 ± 0.9 | 57.8 ± 0.1 | 518 ± 4.2 | 2 ^{c} |
| Opti-Pgp | None | 42.7 ± 1.7 | ND | 264 ± 67 | 11 ^{d} |
| | 1% lipid | 49.3 ± 1.0 | 58.7 ± 0.5 | 567 ± 33 | 5 |

^{a} Temperatures corresponding to the two maxima of the unfolding profiles seen in FIG. 8.
^{b} Number of independent studies.
^{c} Averages ± range are given.
^{d} routinely conducted in 20 mM HEPES, pH 7.6, 50 mM NaCl, 10% glycerol, 0.1% DDM and 5.5 mM DTT. Four studies were conducted in buffers containing 40 mM imidazole, and three experiments were conducted with reduced glycerol (5% instead of 10% glycerol); no significant differences in the Tₘ or ΔH_{cal} were observed under those conditions.

Strikingly, a distinct second unfolding transition appeared at ˜58° C. suggesting sequential unfolding of at least two domains in the protein [55, 56]. It is tempting to assign the higher transition to unfolding of the TMDs which, under these conditions, are expected to reside within the hydrophobic core of the lipid bilayer. This environment may promote the acquisition of a more cooperative and/or more folded structure by providing better aqueous solvent exclusion for the TMDs than detergent, and/or there may be specific lipid-protein interactions which would thermodynamically favor a more folded structure. Other explanations for TMD stabilization are also possible [57, 58]. Titration of Opti-Pgp with lipid showed that the lipid-dependent changes in Tₘ occurred progressively, with an intermediate Tₘ seen at 0.13% lipid (48° C.) and two distinct Tₘ maxima resolving at lipid concentrations ≧0.52% (FIG. 8 C-F). The increase in thermal stability was paralleled by an increase in ATPase activity with increasing lipid concentrations (FIG. 9). Together, the data suggest that an increase in stable ternary structure over the entire Pgp molecule may be responsible for the robust ATPase activity seen when the protein is surrounded by saturating lipid molecules. However, phospholipids also serve as transport substrates of Pgp [59] and we cannot exclude the possibility that some lipid-substrate molecules bound to the drug binding site may promote folding in the manner of chemical chaperones, in addition to hydrophobic interactions at the protein-lipid interface [60].

Previously, human Pgp single-nucleotide polymorphisms (SNPs) that introduce rare codons were suggested to alter the structure of substrate and inhibitor interaction sites by affecting the timing of cotranslational folding and membrane insertion [40, 61, 62, 63]. In these studies, the human MDR1 haplotype consisting of the synonymous polymorphisms C3435T (Ile1145) and C1236T (Gly412) in combination with G2677T, which changes Ala893 to Ser led to reduced Pgp affinity for verapamil and the inhibitor cyclosporine A. Additionally, this haplotype altered susceptibility of the protein to trypsin cleavage [40]. These studies suggested that the tertiary structures of wild-type and the haplotype Pgp differed, which may affect the pharmacokinetics and efficacy of cancer drug treatment [61]. Because of the potential impact of even subtle conformational changes, it was important to confirm that Opti-Pgp retained both substrate specificity and tertiary structure. Trypsin cleavage sites appeared equally accessible in WT- and Opti-Pgp (FIG. 7), suggesting that the two proteins indeed have a similar folded state. This was also corroborated in our DSC study by their similar unfolding temperatures and enthalphies in the absence or presence of lipids (FIGS. 8A-D, Table 2). Interestingly, two of these haplotype codons occur in the homologous positions of the native mouse gene: Ile1141 (ATT) and Ser889 (TCT). It may be noted that ATT and TCT actually represent preferred codons in Pichia yeast (Table 1), in contrast to codons found in human genes. Thus, introduction of these SNPs during codon-optimization of the mouse (or human) gene for Pichia would not be expected to affect cotranslational folding and membrane insertion of Pgp in yeast expression systems.

Finally it is appropriate to comment on the superior optimization procedure proposed in this study. Previous gene optimization procedures aimed to adjust codon usage of the heterologous gene sequence to that of the P. pastoris host either by replacing codons with low usage percentage (<15%) by those with higher usage frequency [21, 64, 65], or, more recently, by simply changing all codons to the most frequently used synonymous codon [66, 67]. Codon analyses, including those offered by commercial sources (e.g. GeneArt, GenScript) were commonly based on the Kazusa codon usage database (http://www.kazusa.or.jp/codon/). Neither the Kazusa database, currently containing 137 coding sequences (CDS's), nor the more complete codon usage table of the P. pastoris ORFeome with 5,313 CDS's that was recently obtained by genome sequencing [23, 29], discriminates between poorly and highly expressed genes. But codon usage in P. pastoris (and in S. cerevisiae) appears significantly more stringent in highly expressed genes, as evident from the larger number of low-frequency codons (Table 1). Furthermore, there are inverted preferences for certain yeast preferred and higher frequency codons (see Table 1 legend), suggesting that preferred codons assigned in the Kazusa database may not always represent the best codon choice for high level expression [19, 21, 68]. The new approach in this study was not only to omit 19 rare codons (<8% frequency) but to completely harmonize the frequency of codons to those of highly expressed P. pastoris genes, and so to maximize translational efficiency by emulating the host's evolutionarily determined codon usage strategy [51, 69].

The present invention provides evidence that substrate specificity and folding were preserved in the gene-optimized Pgp expressed in P. pastoris. Together with transport function, higher protein yield and purity warrant the use of this protein for biophysical studies. Furthermore, the successful gene optimization approach described here may provide a basis for yeast expression of other ABC transporters and membrane proteins, especially in those cases in which poor expression of the native gene have precluded purification efforts [35]. Indeed, preliminary expression analyses of poorer expressers than the mouse Pgp, e.g. the human Pgp (MDR1) or the Cystic Fibrosis Conductance Regulator (CFTR), a protein notorious for its low expression and high turnover in cells [70], suggest that expression levels are increased at least 5-fold compared to the respective WT proteins³). Finally, gene synthesis concurrent with gene optimization may offer a cost effective alternative for expression of proteins identified from genome sequencing projects for which a physical eDNA is not yet available.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Ambudkar S V, Dey S, Hrycyna C A, Ramachandra M, Pastan I, et al. (1999) Biochemical, cellular, and pharmacological aspects of the multidrug transporter. Annu Rev Pharmacal Toxicol 39: 361-398.
2. Gottesman M M, Ling V (2006) The molecular basis of multidrug resistance in cancer: the early years of P-glycoprotein research. FEBS Lett 580: 998-1009.
3. Szakacs G, Paterson J K, Ludwig J A, Booth-Genthe C, Gottesman M M (2006) Targeting multidrug resistance in cancer. Nat Rev Drug Discov 5: 219-234.
4. Sharom F J (2008) ABC multidrug transporters: structure, function and role in chemoresistance. Pharmacogenomics 9: 105-127.
5. Schinkel A H (1999) P-Glycoprotein, a gatekeeper in the blood-brain barrier. Adv Drug Deliv Rev 36: 179-194.
6. Gimenez F, Fernandez C, Mabondzo A (2004) Transport of HIV protease inhibitors through the blood-brain barrier and interactions with the efflux proteins, P-glycoprotein and multidrug resistance proteins. J Acquir Immune Defic Syndr 36: 649-658.
7. Hughes J R (2008) One of the hottest topics in epileptology: ABC proteins. Their inhibition may be the future for patients with intractable seizures. Neurol Res 30: 920-925.
8. Pariante C M (2008) The role of multi-drug resistance p-glycoprotein in glucocorticoid function: studies in animals and relevance in humans. Eur J Pharmaco1583: 263-271.
9. Rees D C, Johnson E, Lewinson 0 (2009) ABC transporters: the power to change. Nat Rev Mol Cell Biol 10: 218-227.
10. Gutmann D A, Ward A, Urbatsch I L, Chang G, van Veen H W (2010) Understanding polyspecificity of multidrug ABC transporters: closing in on the gaps in ABCB1. Trends Biochem Sci 35: 36-42.
11. Aller S G, Yu J, Ward A, Weng Y, Chittaboina S, et al. (2009) Structure of P-glycoprotein reveals a molecular basis for poly-specific drug binding. Science 323: 1718-1722.
12. Urbatsch I L, Beaudet L, Carrier I, Gros P (1998) Mutations in either nucleotide-binding site of P-glycoprotein (MDR3) prevent vanadate trapping of nucleotide at both sites. Biochemistry 37: 4592-4602.
13. Lerner-Marmarosh N, Gimi K, Urbatsch I L, Gros P, Senior A E (1999) Large scale purification of detergent-soluble P-glycoprotein from Pichia pastoris cells and characterization of nucleotide binding properties of wild-type, Walker A, and Walker B mutant proteins. J Biol Chem 274: 34711-34718.
14. Ikemura T (1982) Correlation between the abundance of yeast transfer RNAs and the occurrence of the respective codons in protein genes. Differences in synonymous codon choice patterns of yeast and Escherichia coli with reference to the abundance of isoaccepting transfer RNAs. J Mol Biol 158: 573-597.
15. Hani J, Feldmann H (1998) tRNA genes and retroelements in the yeast genome. Nucleic Acids Res 26: 689-696.
16. Quartley E, Alexandrov A, Mikucki M, Buckner F S, Hol W G, et al. (2009) Heterologous expression of L. major proteins in S. cerevisiae: a test of solubility, purity, and gene recoding. J Struct Funct Genomics 10: 233-247.
17. Novy R, Drott D, Yaeger K, Mierendorf R (2001) Overcoming the codon bias of E. coli for enhanced protein expression. in Novations 12: 1-3.
18. Lombardi A, Bursomanno S, Lopardo T, Traini R, Colombatti M, et al. (2010) Pichia pastoris as a host for secretion of toxic saporin chimeras. FASEB J 24: 253-265.
19. Huang H, Yang P, Luo H, Tang H, Shao N, et al. (2008) High-level expression of a truncated 1,3-1,4-beta-D-glucanase from Fibrobacter succinogenes in Pichia pastoris by optimization of codons and fermentation. Appl Microbial Biotechnol 78: 95-103.
20. Daly R, Hearn M T (2005) Expression of heterologous proteins in Pichia pastoris: a useful experimental tool in protein engineering and production. J Mol Recognit 18: 119-138.
21. Sinclair G, Choy F Y (2002) Synonymous codon usage bias and the expression of human glucocerebrosidase in the methylotrophic yeast, Pichia pastoris. Protein Expr Purif 26: 96-105.
22. Sreekrishna K, Brankamp R G, Kropp K E, Blankenship D T, Tsay J T, et al. (1997) Strategies for optimal synthesis and secretion of heterologous proteins in the methylotrophic yeast Pichia pastoris. Gene 190: 55-62.
23. De Schutter K, Lin Y C, Tiels P, Van Heeke A, Glinka S, et al. (2009) Genome sequence of the recombinant protein production host Pichia pastoris. Nat Biotechnol 27: 561-566.
24. Mattanovich D, Callewaert N, Rouze P, Lin Y C, Graf A, et al. (2009) Open access to sequence: browsing the Pichia pastoris genome. Microb Cell Fact 8: 53.
25. Urbatsch I L, Wilke-Mounts S, Gimi K, Senior A E (2001) Purification and characterization of N-glycosylation mutant mouse and human P-glycoproteins expressed in Pichia pastoris cells. Arch Biochem Biophys 388: 171-177.
26. Dragosits M, Stadlmann J, Albiol J, Baumann K, Maurer M, et al. (2009) The effect of temperature on the proteome of recombinant Pichia pastoris. J Proteome Res 8: 1380-1392.
27. Dragosits M, Stadlmann J, Graf A, Gasser B, Maurer M, et al. (2010) The response to unfolded protein is involved in osmotolerance of Pichia pastoris. BMC Genomics 11: 207.
28. Baumann K, Camicer M, Dragosits M, Graf A B, Stadlmann J, et al. (2010) A multi-level study of recombinant Pichia pastoris in different oxygen conditions. BMC Syst Biol 4: 141.
29. Mattanovich D, Graf A, Stadlmann J, Dragosits M, Redl A, et al. (2009) Genome, secretome and glucose transport highlight unique features of the protein production host Pichia pastoris. Microb Cell Fact 8: 29.
30. Sauer M, Branduardi P, Gasser B, Valli M, Maurer M, et al. (2004) Differential gene expression in recombinant Pichia pastoris analysed by heterologous DNA microarray hybridisation. Microb Cell Fact 3: 17.
31. Johnson B J, Lee J Y, Pickert A, Urbatsch I L (2010) Bile acids stimulate ATP hydrolysis in the purified cholesterol transporter ABCG5/G8. Biochemistry 49: 3403-3411.
32. Vemet T, Dignard D, Thomas D Y (1987) A family of yeast expression vectors containing the phage fl intergenic region. Gene 52: 225-233.
33. Raymond M, Ruetz S, Thomas D Y, Gros P (1994) Functional expression of P-glycoprotein in Saccharomyces cerevisiae confers cellular resistance to the immunosuppressive and antifungal agent FK520. Mol Cell Bio 14: 277-286.
34. Raymond M, Gros P, Whiteway M, Thomas D Y (1992) Functional complementation of yeast step6 by a mammalian multidrug resistance MDR gene. Science 256: 232-234.
35. Chloupkova M, Pickert A, Lee J Y, Souza S, Trinh Y T, et al. (2007) Expression of 25 human ABC transporters in the yeast Pichia pastoris and characterization of the purified ABCC3 ATPase activity. Biochemistry 46: 7992-8003.
36. Urbatsch I L, Sankaran B, Weber J, Senior A E (1995) P-glycoprotein is stably inhibited by vanadate-induced trapping of nucleotide at a single catalytic site. J Biol Chem 270: 19383-19390.
37. Urbatsch I L, Tyndall G A, Tombline G, Senior A E (2003) P-glycoprotein catalytic mechanism: studies of the ADP-vanadate inhibited state. J Biol Chem 278: 23171-23179.
38. Lin-Cereghino G P, Godfrey L, de la Cruz B J, Johnson S, Khuongsathiene S, et al. (2006) Mxrlp, a key regulator of the methanol utilization pathway and peroxisomal genes in Pichia pastoris. Mol Cell Biol 26: 883-897.
39. Kotisreekrishna K (1998) Methods of Enzymology.
40. Kimchi-Sarfaty C, Oh J M, Kim I W, Sauna Z E, Calcagno A M, et al. (2007) A “silent” polymorphism in the MDR1 gene changes substrate specificity. Science 315: 525-528.
41. Urbatsch I L, Julien M, Carrier I, Rousseau M E, Cayrol R, et al. (2000) Mutational analysis of conserved carboxylate residues in the nucleotide binding sites of P-glycoprotein. Biochemistry 39: 14138-14149.
42. Urbatsch I L, Gimi K, Wilke-Mounts S, Lerner-Marmarosh N, Rousseau M E, et al. (2001) Cysteines 431 and 1074 are responsible for inhibitory disulfide cross-linking between the two nucleotide-binding sites in human P-glycoprotein. J Biol Chem 276: 26980-26987.
43. Chen Y H, Yang J T, Martinez H M (1972) Determination of the secondary structures of proteins by circular dichroism and optical rotatory dispersion. Biochemistry 11: 4120-4131.
44. Nuti S L, Rao U S (2002) Proteolytic Cleavage of the Linker Region of the Human Pglycoprotein Modulates Its ATPase Function. J Biol Chem 277: 29417-29423.
45. Cereghino G P, Cregg J M (1999) Applications of yeast in biotechnology: protein production and genetic analysis. Curr Opin BiotechnollO: 422-427.
46. Tombline G, Bartholomew L A, Urbatsch I L, Senior A E (2004) Combined mutation of catalytic glutamate residues in the two nucleotide binding domains of P-glycoprotein generates a conformation that binds ATP and ADP tightly. J Biol Chem 279: 31212-31220.
47. Tombline G, Senior A E (2005) The occluded nucleotide conformation of p-glycoprotein. J Bioenerg Biomembr 37: 497-500.
48. Urbatsch I L, Gimi K, Wilke-Mounts S, Senior A E (2000) Conserved walker A Ser residues in the catalytic sites of P-glycoprotein are critical for catalysis and involved primarily at the transition state step. J Biol Chem 275: 25031-25038.
49. Lee J Y, Urbatsch I L, Senior A E, Wilkens S (2002) Projection structure of P-glycoprotein by electron microscopy. Evidence for a closed conformation of the nucleotide binding domains. J Biol Chem 277: 40125-40131.
50. Lee J Y, Urbatsch I L, Senior A E, Wilkens S (2008) Nucleotide-induced structural changes in P-glycoprotein observed by electron microscopy. J Biol Chem 283: 5769-5779.
51. Komar A A (2009) A pause for thought along the co-translational folding pathway. Trends Biochem Sci 34: 16-24.
52. Reinau M E, Otzen D E (2009) Stability and structure of the membrane protein transporter Ffh is modulated by substrates and lipids. Arch Biochem Biophys 492: 48-53.
53. Soubias O, Niu S L, Mitchell D C, Gawrisch K (2008) Lipid-rhodopsin hydrophobic mismatch alters rhodopsin helical content. J Am Chem Soc 130: 12465-12471.
54. Ortega A, Santiago-Garcia J, Mas-Oliva J, Lepock J R (1996) Cholesterol increases the thermal stability of the Ca2+/Mg(2+)-ATPase of cardiac microsomes. Biochim Biophys Acta 1283: 45-50.
55. Jaenicke R, Lilie H (2000) Folding and association of oligomeric and multimeric proteins. Adv Protein Chem 53: 329-401.
56. Privalov P L (1982) Stability of proteins. Proteins which do not present a single cooperative system. Adv Protein Chem 35: 1-104.
57. Brouillette C G, Muccio D D, Finney T K (1987) pH dependence of bacteriorhodopsin thermal unfolding. Biochemistry 26: 7431-7438.
58. Stowell M H, Rees D C (1995) Structure and stability of membrane proteins. Adv Protein Chem 46: 279-311.
59. Eckford P D, Sharom F J (2009) ABC efflux pump-based resistance to chemotherapy drugs. Chem Rev 109: 2989-3011.
60. Callaghan R, Berridge G, Ferry D R, Higgins C F (1997) The functional purification of Pglycoprotein is dependent on maintenance of a lipid-protein interface. Biochim Biophys Acta 1328: 109-124.
61. Kimchi-Sarfaty C, Marple A H, Shinar S, Kimchi A M, Scavo D, et al. (2007) Ethnicityrelated polymorphisms and haplotypes in the human ABCB1 gene. Pharmacogenomics 8: 29-39.
62. Sauna Z E, Kimchi-Sarfaty C, Ambudkar S V, Gottesman M M (2007) Silent polymorphisms speak: how they affect pharmacogenomics and the treatment of cancer. Cancer Res 67:9609 9612.
63. Tsai C J, Sauna Z E, Kimchi-Sarfaty C, Ambudkar S V, Gottesman M M, et al. (2008) Synonymous mutations and ribosome stalling can lead to altered folding pathways and distinct minima. J Mol Biol 383: 281-291.
64. Su Z, Wu X, Feng Y, Ding C, Xiao Y, et al. (2007) High level expression of human endostatin in Pichia pastoris using a synthetic gene construct. Appl Microbial Biotechnol 73: 1355-1362.
65. Teng D, Fan Y, Yang Y L, Tian Z G, Luo J, et al. (2007) Codon optimization of Bacillus licheniformis beta-1,3-1,4-glucanase gene and its expression in Pichia pastoris. Appl Microbial Biotechnol 74: 1074-1083.
66. Lee S G, Koh H Y, Han S J, Park H, Na D C, et al. (2010) Expression of recombinant endochitinase from the Antarctic bacterium, Sanguibacter antarcticus KOPRI 21702 in Pichia pastoris by codon optimization. Protein Expr Purif71: 108-114.
67. Scholz C, Parcej D, Ejsing C S, Robenek H, Urbatsch I L, et al. (2011) Transporter associated with antigen processing (TAP) is modulated by lipids. J Biol. Chem.
68. Zhao X, Huo K K, Li Y Y (2000) [Synonymous codon usage in Pichia pastoris]. Sheng Wu Gong Cheng Xue Bao 16: 308-311.
69. Lavner Y, Kotlar D (2005) Codon bias as a factor in regulating expression via translation rate in the human genome. Gene 345: 127-138.
70. Farinha C M, Penque D, Roxo-Rosa M, Lukacs G, Dormer R, et al. (2004) Biochemical methods to assess CFTR expression and membrane localization. J Cyst Fibros 3 Suppl 2: 73-77.

What is claimed is:

1. A method of codon optimization to increase protein production comprising the steps of:
providing a target gene, wherein the expression of the target gene is to be optimized;
determining one or more low-frequency codons in the target gene;
providing a codon usage frequency table comprising one or more high-frequency codons, wherein the codon usage frequency table is based on a set of highly expressed native genes comprising ACO1 (Pas_chr1-3_{—}0104), ACS1 (Pas_chr2-1_{—}0767), AOX1 (Pas_chr4_{—}0821, PPU96967); CAT2 (Pas_chr3_{—}0069), CCP1 (Pas_chr2-2_{—}0127), CDC19 (Pas_chr2-1_{—}0769), CTA1 (Pas_chr2-2_{—}0131), ENOL (Pas_chr3_{—}0082), FBA1 (Pas_chr1-1_{—}0072), FDH1 (Pas_chr3_{—}0932), FLD1 (AF066054), GDH3 (Pas_chr1-1_{—}0107), GPM1 (Pas_chr3_{—}0826), GUT2 (Pas_chr3_{—}0579), HSP82 (Pas_chr1-4_{—}0130), ICL1 (Pas_chr1-4_{—}0338), ILV5 (Pas_chr1-1_{—}0432), KAR2 (Pas_chr2-1_{—}0140, AY965684), MDH1 (Pas_chr2-1_{—}0238), MET6 (Pas_chr2-1_{—}0160, AY601648), PDI1 (Pas_chr4_{—}0844, AJ302014), PGK1 (Pas_chr1-4_{—}0292), PIL1 (Pas_chr1-4_{—}0569), RPP0 (Pas_chr1-3_{—}0068), SSA3 (Pas_chr3_{—}0230), SSB2 (Pas_chr3_{—}0731), SSC1 (Pas_chr3_{—}0365), TDH3 (Pas_chr2-1_{—}0437, also called GAP, PPU62648), TEF2 (Pas_FragB_{—}0052, AY219033), YEF3 (Pas_chr4_{—}0038, also called TEF3, and AB018536);
replacing each of the one or more low-frequency codons in the target gene with a corresponding high-frequency codons that code for the same amino acid; and
harmonizing a distribution of codon frequencies to those of the set of highly expressed native gene over an open reading frame in the target gene to form an optimized gene, wherein the optimized gene encodes an amino acid sequence identical to the respective wild-type (native) amino acid sequence.

2. The method of claim 1, wherein the one or more low-frequency codons vary at less than ±5% frequency.

3. The method of claim 1, wherein the one or more high-frequency codons vary at less than ±10% frequency.

4. The method of claim 1, wherein the target gene codes for a P-glycoprotein, the mouse MDR3 (mdr1a, abcb1a gene).

5. The method of claim 1, wherein the target gene codes for a P-glycoprotein, the human MDR1 (ABCB1 gene).

6. The method of claim 1, wherein the optimized gene produces at least a 18, 17, 16, 15, 14, 13, 12, 11, 10, 9, 8, 7, 6, 5, 4 or 3 fold increase in the functional protein compared to the expression of a native gene.

7. An optimized cDNA encoding an optimized gene made by the method of codon optimization comprising the steps of:
providing a target gene, wherein the expression of the target gene is to be optimized;
determining one or more low-frequency codons in the target gene;
providing a codon usage frequency table comprising one or more high-frequency codons, wherein the codon usage frequency table is based on a set of highly expressed native genes comprising ACO1 (Pas_chr1-3_{—}0104), ACS1 (Pas_chr2-1_{—}0767), AOX1 (Pas_chr4_{—}0821, PPU96967); CAT2 (Pas_chr3_{—}0069), CCP1 (Pas_chr2-2_{—}0127), CDC19 (Pas_chr2-1_{—}0769), CTA1 (Pas_chr2-2_{—}0131), ENOL (Pas_chr3_{—}0082), FBA1 (Pas_chr1-1_{—}0072), FDH1 (Pas_chr3_{—}0932), FLD1 (AF066054), GDH3 (Pas_chr1-1_{—}0107), GPM1 (Pas_chr3_{—}0826), GUT2 (Pas_chr3_{—}0579), HSP82 (Pas_chr1-4_{—}0130), ICL1 (Pas_chr1-4_{—}0338), ILV5 (Pas_chr1-1_{—}0432), KAR2 (Pas_chr2-1_{—}0140, AY965684), MDH1 (Pas_chr2-1_{—}0238), MET6 (Pas_chr2-1_{—}0160, AY601648), PDI1 (Pas_chr4_{—}0844, AJ302014), PGK1 (Pas_chr1-4_{—}0292), PIL1 (Pas_chr1-4_{—}0569), RPP0 (Pas_chr1-3_{—}0068), SSA3 (Pas_chr3_{—}0230), SSB2 (Pas_chr3_{—}0731), SSC1 (Pas_chr3_{—}0365), TDH3 (Pas_chr2-1_{—}0437, also called GAP, PPU62648), TEF2 (Pas_FragB_{—}0052, AY219033), YEF3 (Pas_chr4_{—}0038, also called TEF3, and AB018536);
replacing each of the one or more low-frequency codons in the target gene with a corresponding high-frequency codons that code for the same amino acid;
harmonizing a distribution of codon frequencies to those of the set of highly expressed native gene over an open reading frame in the target gene to form an optimized gene, wherein the optimized gene encodes an amino acid sequence identical to the respective wild-type (native) amino acid sequence; and
forming an optimized cDNA encoding an optimized gene.

8. The optimized cDNA encoding an optimized gene of claim 7, wherein the optimized cDNA encodes a gene-optimized Mdr3 P-glycoprotein (opti-mdr3, mouse abcb1a gene).

9. The optimized cDNA encoding an optimized gene of claim 7, wherein the optimized cDNA encodes a gene-optimized MDR1 P-glycoprotein (opti-MDR1, human ABCB1 gene).

10. An expression optimized cell to increase production of a functional protein comprising:
a cell containing an optimized cDNA encoding an optimized gene, wherein the optimized cDNA encoding an optimized gene is made by the method of codon optimization comprising the steps of:
providing a target gene, wherein the expression of the target gene is to be optimized;
determining one or more low-frequency codons in the target gene;
providing a codon usage frequency table comprising one or more high-frequency codons, wherein the codon usage frequency table is based on a set of highly expressed native genes comprising ACO1 (Pas_chr1-3_{—}0104), ACS1 (Pas_chr2-1_{—}0767), AOX1 (Pas_chr4_{—}0821, PPU96967); CAT2 (Pas_chr3_{—}0069), CCP1 (Pas_chr2-2_{—}0127), CDC19 (Pas_chr2-1_{—}0769), CTA1 (Pas_chr2-2_{—}0131), ENOL (Pas_chr3_{—}0082), FBA1 (Pas_chr1-1_{—}0072), FDH1 (Pas_chr3_{—}0932), FLD1 (AF066054), GDH3 (Pas_chr1-1_{—}0107), GPM1 (Pas_chr3_{—}0826), GUT2 (Pas_chr3_{—}0579), HSP82 (Pas_chr1-4_{—}0130), ICL1 (Pas_chr1-4_{—}0338), ILV5 (Pas_chr1-1_{—}0432), KAR2 (Pas_chr2-1_{—}0140, AY965684), MDH1 (Pas_chr2-1_{—}0238), MET6 (Pas_chr2-1_{—}0160, AY601648), PDI1 (Pas_chr4_{—}0844, AJ302014), PGK1 (Pas_chr1-4_{—}0292), PIL1 (Pas_chr1-4_{—}0569), RPP0 (Pas_chr1-3_{—}0068), SSA3 (Pas_chr3_{—}0230), SSB2 (Pas_chr3_{—}0731), SSC1 (Pas_chr3_{—}0365), TDH3 (Pas_chr2-1_{—}0437, also called GAP, PPU62648), TEF2 (Pas_FragB_{—}0052, AY219033), YEF3 (Pas_chr4_{—}0038, also called TEF3, and AB018536);
replacing each of the one or more low-frequency codons in the target gene with a corresponding high-frequency codons that code for the same amino acid;
harmonizing a distribution of codon frequencies to those of the set of highly expressed native gene over an open reading frame in the target gene to form an optimized gene, wherein the optimized gene encodes an amino acid sequence identical to the respective wild-type (native) amino acid sequence; and
forming an optimized cDNA encoding an optimized gene.

11. The method of claim 10, wherein the cell is a yeast cell.

12. The method of claim 10, wherein the cell is a Pichia pastoris cell or a Saccharomyces cerevisiae cell.

13. The Saccharomyces cerevisiae strain expressing high levels of mouse P-glycoprotein, mouse opti-Pgp (abcb1a gene) made by the method of claim 12.

14. The Pichia pastoris strain expressing high levels of mouse P-glycoprotein, mouse opti-Pgp (abcb1a gene) made by the method of claim 12.

15. The Pichia pastoris strain expressing high levels of human P-glycoprotein, human opti-MDR1 (ABCB1 gene) made by the method of claim 12.

16. The method of claim 10, wherein the optimized gene produces at least a 18, 17, 16, 15, 14, 13, 12, 11, 10, 9, 8, 7, 6, 5, 4 or 3 fold increase in the functional protein compared to the expression of a native gene.

17. An apparatus for codon optimization to increase protein production, the apparatus comprising;
an interface to a codon set of 30 native genes that are highly expressed in P. pastoris, wherein the codon set of 30 native genes comprises ACO1 (Pas_chr1-3_{—}0104), ACS1 (Pas_chr2-1_{—}0767), AOX1 (Pas_chr4_{—}0821, PPU96967); CAT2 (Pas_chr3_{—}0069), CCP1 (Pas_chr2-2_{—}0127), CDC19 (Pas_chr2-1_{—}0769), CTA1 (Pas_chr2-2_{—}0131), ENOL (Pas_chr3_{—}0082), FBA1 (Pas_chr1-1_{—}0072), FDH1 (Pas_chr3_{—}0932), FLD1 (AF066054), GDH3 (Pas_chr1-1_{—}0107), GPM1 (Pas_chr3_{—}0826), GUT2 (Pas_chr3_{—}0579), HSP82 (Pas_chr1-4_{—}0130), ICL1 (Pas_chr1-4_{—}0338), ILV5 (Pas_chr1-1_{—}0432), KAR2 (Pas_chr2-1_{—}0140, AY965684), MDH1 (Pas_chr2-1_{—}0238), MET6 (Pas_chr2-1_{—}0160, AY601648), PDI1 (Pas_chr4_{—}0844, AJ302014), PGK1 (Pas_chr1-4_{—}0292), PILI (Pas_chr1-4_{—}0569), RPP0 (Pas_chr1-3_{—}0068), SSA3 (Pas_chr3_{—}0230), SSB2 (Pas_chr3_{—}0731), SSC1 (Pas_chr3_{—}0365), TDH3 (Pas_chr2-1_{—}0437, also called GAP, PPU62648), TEF2 (Pas_FragB_{—}0052, AY219033), and YEF3 (Pas_chr4_{—}0038, also called TEF3, AB018536);
a memory; and
a processor communicably connected to the interface and the memory, wherein the processor produces a codon usage frequency table from the codon set of 30 native genes and provides a set of low-frequency codons and a set of high-frequency codons.

18. The apparatus of claim 17, wherein the processor optimizes the expression of the target gene by using the codon usage frequency table to replace each low-frequency codon in a target gene with a corresponding high-frequency codon from the codon usage frequency table that code for the same amino acid and harmonizing the a distribution of codon frequencies to those of the set of highly expressed native gene over an open reading frame in the target gene to form an optimized gene, wherein the optimized gene encodes an amino acid sequence identical to the respective wild-type (native) amino acid sequence.

19. A codon usage frequency table made by the apparatus in claim 17.
